(19) United States
(12) Patent Application Publication
(10) Pub. No.: US 2023/0149289 A1
(43) Pub. Date: May 18, 2023

(54) COSMETIC COMPOSITION FOR FORMING A FILM HAVING ELEVATED ELASTICITY AND EXTENSIBILITY

(71) Applicant: Covestro Deutschland AG, Leverkusen (DE)

(72) Inventors: Laurence Pottie, Köln (DE); Sophie Viala, Köln (DE)

(21) Appl. No.: 17/801,541

(22) PCT Filed: Feb. 24, 2021
(86) PCT No.: PCT/EP2021/054533

§ 371 Date: Aug. 23, 2022

(30) Foreign Application Priority Data
Mar. 2, 2020 (WO) 20160302.4

(51) Int. Cl. A61K 8/87; A61K 8/34; A61Q 19/10; A61Q 5/02; A61Q 3/02

(52) U.S. Cl. CPC A61K 8/87; A61K 8/345; A61Q 19/10; A61Q 5/02; A61Q 3/02; A61K 2800/95

(57) ABSTRACT

The present invention relates to a cosmetic composition comprising at least one aqueous polyurethane dispersion and 0.1 to 7.5% by weight, based on the at least one polyurethane dispersion, of a mixture comprising at least one alkanediol, to a process for production thereof, to the use thereof for application to the skin and/or hair, preferably on facial skin, and to a cosmetic process for cleansing and caring for the skin and/or hair and/or nails and/or for applying a decorative effect to the skin and/or hair and/or nails, comprising applying the cosmetic composition according to the invention to the skin and/or hair and/or nails and optionally thereafter removing the composition from the skin and/or hair and/or nails.

The present invention relates to a cosmetic composition comprising at least one aqueous polyurethane dispersion and 0.1 to 7.5% by weight, based on the at least one aqueous polyurethane dispersion, of a mixture comprising at least one alkanediol, to a process for production thereof, to the use thereof for application to the skin, the hair and/or nails, and to a cosmetic process for cleansing and caring for the skin and/or hair and/or nails, to applying a decorative effect to the skin and/or hair and/or nails, comprising applying the cosmetic composition according to the invention to the skin and/or hair and/or nails and optionally thereafter removing the composition from the skin and/or hair and/or nails.

In the context of the present invention, cosmetic compositions are those which may be used for cleansing and/or caring for the skin and/or hair and/or nails.

A skin care product is a cosmetic composition for application to the skin, such as in particular to the face and/or other parts of the body. The skin care product serves in particular to protect against skin changes such as skin aging, dehydration or the like. Skin care products are intended to restore and maintain the skin in its normal physiological state. The horny layers are supported in their natural regenerative ability when damaged, i.e. the upper horny layers are moistened and protected. In addition, the permeability properties of the skin barrier should be restored and skin renewal should be supported. A skin care product should additionally leave the skin feeling supple after application. Furthermore, it is desirable for a skin-care product to form a sufficiently elastic film to achieve a skin-tightening effect. It is also desirable that corresponding skin care creams form a sufficiently elastic film that can adapt to body movements during sport.

Cosmetic compositions are also used in sunscreen creams. For years, tanned skin has been synonymous with attractive, healthy, athletic and successful people. To achieve this, people expose their skin to the sun's rays. However, the sun's rays have a damaging effect on the skin, as they penetrate the skin at different depths depending on the wavelength. The shorter-wave radiation in the UVB range (wavelength 280 to 320 nm) reaches the uppermost skin layer. Rays in the UVB range cause sunburn and are responsible for an increased risk of skin cancer. The longer-wave UVA rays (wavelength 320 to 400 nm) penetrate deeper skin layers. They lead to damage to the collagen and elastin fibers which are of essential importance for the structure and firmness of the skin. This also leads to premature skin aging due to the formation of wrinkles and fine lines and irregular relief of the skin, etc. To protect the skin against solar radiation, light protection filter substances have been developed, i.e. UVA and UVB filters, contained in the form of positive lists such as Annex VI, last update of regulation (EC)No 1223/2009 from 05/08/2019, which are used in cosmetic and dermatological compositions.

Sun protection products are often used on vacation or during leisure time at the beach or during sports activities where the body is in contact with water or perspiration and is in motion at the same time. It is important in this case that the sun protection product has a high degree of elasticity after application to the skin, so that it can adapt to body movements.

Furthermore, cosmetic compositions can also be used for care and/or cleansing of the skin, in particular facial skin, in the form of so-called “peel-off masks”. In this case, the cosmetic composition is applied to the skin, particularly facial skin. After the product dries, a coherent film forms on the skin, which remains on the skin for a certain amount of time in order to develop the caring effect of the cosmetic composition. The cosmetic composition in the form of the film can then be removed ideally as a whole. For this application, it is important that a sufficiently elastic and stable film is formed that can be removed as a whole after use.

A decorative cosmetic composition is used for the decorative, in particular colorful design of human skin, mucosa, semi-mucosa and hair, in particular eyelids, eyebrows and/or nails. The decorative effect is achieved by at least one effect-generating component. The decorative composition according to the invention can, for example, be a face make-up (foundation), a tinted (day) cream, a blush, a rouge, a mascara, an eyeliner, a kajal, an eye shadow, a lipstick, a lip gloss, a nail polish. These special cosmetic formulations are used to change the color or to apply make-up to the body, for example to cover up dark rings under the eyes, an uneven complexion or other skin imperfections such as redness, spots, wrinkles or pimples, thus giving the user a more aesthetic appearance. The aforementioned list of decorative products is of course non-limiting.

The decorative cosmetic compositions suitably comprise one or more dyes selected, for example, from the group consisting of soluble dyes, inorganic pigments such as iron oxides, chromium oxides, ultramarine, manganese violet, organic pigments and nacre. Depending on the formulation, such decorative cosmetic compositions may consist of up to 80% by weight of colorants and fillers, based on the total weight of the composition.

To create and stabilize versatile hairstyles, products known as hair styling products are used. Hair setting products are mostly available in the form of mousses or hairsprays. Mousses and hairsprays hardly differ in their composition but rather in their application. Mousses are applied to damp hair as an aid in modeling the hairstyle. In contrast, hairsprays are sprayed onto dry, ready-styled hair to fix the hairstyle. In addition to hair sprays and mousses, hair setting gels are also available. So-called “leave-on conditioners” are also used to care for hair.

In the case of hairsprays and mousses, the means of fixing or styling the hairstyle usually takes the form of aerosol containers, squeeze bottles or preparations sprayable by pumping, spraying or foaming devices, which consist of an alcoholic or aqueous-alcoholic solution of film-forming natural or synthetic polymers. These polymers may be selected from the group of non-ionic, cationic, amphoteric or anionic polymers. In the case of hair setting gels, the preparations described above are adjusted to an acceptable viscosity using conventional thickeners.

Document EP 2 271 307 A2 discloses a skin-care composition for application to the skin, comprising specific polyurethanes and the use of the specified polyurethanes for producing skin-care products. It is further disclosed that this skin care composition has the advantage of forming a pleasant, non-sticky and non-greasy protective film after spreading on the skin.

EP 2 271 306 A1 describes a decorative cosmetic composition comprising specific polyurethanes or aqueous dispersions thereof and constituents providing decorative effects, which is highly comfortable to wear, in particular reduced tackiness, has high resistance, in particular to water, and improved gloss properties. Furthermore, the aqueous polyurethane dispersions used according to this document have a comparatively low viscosity, such that they can easily be incorporated into cosmetic compositions for decorative purposes.

Document EP 2 271 305 A1 discloses a sunscreen composition for application to the skin, comprising specific polyurethanes and the use of said polyurethanes for the production of sunscreen products, wherein said composition generates a cosmetic or dermatological sunscreen composition and has excellent water resistance.

The object of the present invention is to provide a cosmetic composition which, when used in a skin care product, a skin cleansing product, a decorative product, a sunscreen cream or a product for application to the skin and/or hair and/or nails, leaves a smooth feeling after application and forms a sufficiently elastic film, which in particular produces a skin-tightening effect and can therefore adapt well to body movements during sport. When used on hair, the elevated elasticity of the film produced should result in an improved property profile, in particular should result in less flaking, i.e. the formation of white particles when the film breaks on the hair, and at the same time allowing natural hair movement (“bounce”). Furthermore, the styling, i.e. the hold of the hairstyle, should be possible over a longer period of time. Furthermore, it should also be possible to use the cosmetic composition in so-called “peel-off masks” and ensure that, after application, it is possible to remove the cosmetic composition in the form of the film from the skin, in particular facial skin, as far as possible as a whole A further object of the present invention is to provide a corresponding composition that forms a film which exhibits a particularly high elongation without tearing.

These objects are achieved according to the invention by a cosmetic composition comprising at least one aqueous polyurethane dispersion and 0.1 to 7.5% by weight, based on the at least one aqueous polyurethane dispersion, of a mixture comprising at least one alkanediol.

Furthermore, the objects are achieved by a process for the preparation thereof and the use thereof of a cosmetic composition according to the invention for application to the skin and/or hair and/or nails, preferably on facial skin, preferably forming a film on the skin and/or hair and/or nails after application, and by a cosmetic process for cleansing and caring for the skin and/or hair and/or nails and/or for applying a decorative effect to the skin and/or hair and/or nails, comprising applying the cosmetic composition according to the invention to the skin and/or hair and/or nails and optionally thereafter removing the composition from the skin and/or hair and/or nails.

The present invention is described in detail hereinbelow.

The present invention relates to a cosmetic composition comprising at least one aqueous polyurethane dispersion and 0.1 to 7.5% by weight, based on the at least one aqueous polyurethane dispersion, of a mixture comprising at least one alkanediol.

The cosmetic composition according to the invention comprises at least one aqueous polyurethane dispersion.

The at least one polyurethane present in the cosmetic composition according to the invention is preferably obtainable by reacting one or more water-insoluble, non-water-dispersible, isocyanate-functional polyurethane prepolymers A) with one or more amino-functional compounds B).

More preferably, the at least one polyurethane present according to the invention may be obtained by reacting one or more isocyanate-functional polyurethane prepolymers A), which essentially have neither ionic nor ionogenic groups, with one or more amino-functional compounds B).

In the context of the invention, the term “water-insoluble, non-water-dispersible polyurethane prepolymer” means in particular that the water solubility of the prepolymer used in accordance with the invention at 23° C. is less than 10 g/liter, preferably less than 5 g/liter, and the prepolymer at 23° does not result in a sedimentation-stable dispersion in water, especially deionized water. In other words, the prepolymer settles out when an attempt is made to disperse it in water.

Preferably, the polyurethane prepolymer a) used in accordance with the invention has terminal isocyanate groups, meaning that the isocyanate groups are at the chain ends of the prepolymer. All chain ends of a polymer particularly preferably have isocyanate groups.

Furthermore, the polyurethane prepolymer A) used in accordance with the invention preferably has essentially neither ionic nor ionogenic groups, i.e. groups capable of forming ionic groups, i.e. the content of ionic and ionogenic groups is expediently below 15 milliequivalents per 100 g of polyurethane prepolymer A), preferably below 5 milliequivalents, particularly preferably less than one milliequivalent and especially preferably less than 0.1 milliequivalent per 100 g of polyurethane prepolymer A).

The amino-functional compounds B) are preferably selected from primary and/or secondary amines and/or diamines More particularly, the amino-functional compounds B) comprise at least one diamine. The amino-functional compounds B) are preferably selected from amino-functional compounds B2) having an ionic or ionogenic group, and amino-functional compounds B1) having no ionic or ionogenic group.

In a particularly preferred embodiment of the invention, the amino-functional compounds B) comprise at least one amino-functional compound B2) having ionic and/or ionogenic, i.e. ion-forming, groups. Particular preference is given to using the sulfonate or sulfonic acid group, more preferably the sodium sulfonate group, as the ionic and/or ionogenic group.

In a further preferred embodiment of the invention, the amino-functional compounds B) comprise both amino-functional compounds B2) having ionic and/or ionogenic groups and amino-functional compounds B1) having no ionic or ionogenic group.

In the context of the invention, polyurethanes are accordingly polymeric compounds having at least two, preferably at least three, urethane group-containing repeat units:

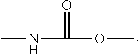

According to the invention, such polyurethanes are also included which, due to the production process, also comprise repeat units containing urea groups:

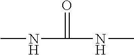

as are formed in particular during the reaction of the isocyanate-terminated prepolymers A) with the amino-functional compounds B).

The cosmetic compositions according to the invention are preferably water-containing, i.e. aqueous compositions in which the polyurethane is dispersed, i.e. largely undissolved. In general, alongside other liquid media that are optionally present, for example solvents, water forms the main constituent, i.e. preferably >50% by weight of the dispersion medium, based on the total amount of the liquid dispersion medium in the cosmetic compositions according to the invention, and possibly even the sole liquid dispersion medium.

The at least one polyurethane present in the cosmetic composition according to the invention is preferably added to the compositions cited as aqueous dispersions.

The water-insoluble and non-water-dispersible, isocyanate-functional polyurethane prepolymers used according to the invention have essentially neither ionic nor ionogenic groups. The water insolubility or lack of dispersibility in water relates to deionized water without addition of surfactants. In the context of the present invention, this means that the proportion of ionic and/or ionogenic groups, such as anionic groups in particular, such as carboxylate or sulfate, or of cationic groups is less than 15 milliequivalents per 100 g of polyurethane prepolymer A), preferably less than 5 milliequivalents, particularly preferably less than one milliequivalent and especially preferably less than 0.1 milliequivalents per 100 g of polyurethane prepolymer A) in each case.

In the case of acidic ionic and/or ionogenic groups, the acid number of the prepolymer is appropriately below 30 mg KOH/g of prepolymer, preferably below 10 mg KOH/g of prepolymer. The acid number indicates the mass of potassium hydroxide in milligrams required to neutralize 1 g of the sample to be examined (measurement to DIN EN ISO 211). The neutralized acids, i.e. the corresponding salts, naturally have no or a reduced acid number. What is crucial here in accordance with the invention is the acid number of the corresponding free acid.

The prepolymers A) used to produce the polyurethanes used according to the invention are preferably obtainable by reacting one or more polyols selected from the group consisting of polyether polyols, polycarbonate polyols, polyether-polycarbonate polyols, polyester polyols and combinations thereof, and at least one polyisocyanate, as explained in more detail below.

Accordingly, the at least one polyurethane present in the cosmetic composition according to the invention comprises, due to the prepolymer A) used for its preparation, preferably at least one sequence selected from the group consisting of: polyether, polycarbonate, polyether-polycarbonate and/or polyester sequences. According to the invention, this means in particular that the at least one polyurethane has ether group and/or carbonate group and/or ester group repeat units. The at least one polyurethane may comprise, for example, exclusively polyether sequences or exclusively polycarbonate sequences or exclusively polyester sequences. However, it may also have both polyether and polycarbonate sequences, as are formed, for example, in the preparation of polycarbonate polyols using polyether diols, as will be described in detail below. In addition, the at least one polyurethane may have polyether-polycarbonate sequences which result from the use of polyether-polycarbonate polyols, as described in more detail below.

Particularly preferred polyurethanes are obtained using polymeric polyether polyols and/or polymeric polycarbonate polyols and/or polyether-polycarbonate polyols or polyester polyols, each having number-average molecular weights of preferably about 400 to about 6000 g/mol. This molecular weight specification and the following molecular weight specifications are each determined by gel permeation chromatography against a polystyrene standard in tetrahydrofuran at 23° C.

Their use in the production of the polyurethanes or polyurethane prepolymers leads to the formation of corresponding polyether, polycarbonate, polyether-polycarbonate sequences and/or polyester sequences in the polyurethanes with a corresponding molecular weight of these sequences as a result of reaction with polyisocyanates. According to the invention, particular preference is given to polyurethanes obtained from polymeric polyether polyols and/or polymeric polycarbonate polyols and/or polyether-polycarbonate polyols or polyester polyols, in particular polymeric polyether diols and/or polymeric polycarbonate diols and/or polyether-polycarbonate diols or polyester diols, each having a linear structure.

The polyurethanes according to the invention are preferably substantially linear molecules, but may also be branched, although this is less preferred.

The number-average molecular weight of the at least one polyurethane preferably used according to the invention is, for example, from about 1000 to 200 000 g/mol, preferably from 5000 to 150 000 g/mol.

The at least one polyurethane or corresponding aqueous polyurethane dispersions to be used by way of preference according to the invention can be obtained by producing
A) isocyanate-functional prepolymers from
A1) organic polyisocyanates,
A2) polymeric polyols, preferably having number-average molecular weights of 400 to 8000 g/mol, determined by gel permeation chromatography compared to a polystyrene standard in tetrahydrofuran at 23° C., more preferably 400 to 6000 g/mol and particularly preferably 600 to 3000 g/mol, and OH functionalities of preferably 1.5 to 6, more preferably 1.8 to 3, particularly preferably 1.9 to 2.1,
A3) optionally hydroxy-functional compounds having molecular weights of preferably 62 to 399 g/mol, and
A4) optionally non-ionic hydrophilizing agents, and
B) some or all of the free NCO groups thereof are then reacted with one or more amino-functional compounds B), such as primary and/or secondary amines and/or diamines.

The polyurethanes used according to the invention are preferably dispersed in water before, during or after step B).

Particular preference is given in step B) to the reaction with a diamine or two or more diamines with chain lengthening. In addition, monofunctional amines may be added as chain terminators to control the molecular weight.

As component B), in particular, amines may be used having no ionic or ionogenic groups, such as anionically hydrophilizing groups (component B1), and amines may be used having ionic or ionogenic groups, such as in particular anionically hydrophilizing groups (component B2).

Preferably, in step B) of the reaction of the prepolymer, a mixture of component B1) and component B2) is reacted. The use of component B1) can result in formation of a high molar mass without a rise in the viscosity of the isocyanate-functional prepolymer prepared beforehand to a degree that would be a barrier to processing. By using the combination of components B1) and B2), an optimal balance can be achieved between hydrophilicity and chain length and thus, in particular, a pleasant skin feel.

The at least one polyurethane used according to the invention preferably has anionic groups, preferably sulfonate groups. These anionic groups are introduced into the polyurethanes used according to the invention via the amine component B2) reacted in step B). The polyurethanes used in accordance with the invention optionally additionally include non-ionic components for hydrophilization. Particularly preferably, the polyurethanes used in accordance with the invention, for hydrophilization, comprise exclusively sulfonate groups which are introduced into the polyurethane via corresponding diamines as component B2).

In order to achieve good sedimentation stability, the number-average particle size of the specific polyurethane dispersions is preferably less than 750 nm, particularly preferably less than 500 nm, determined in each case by means of laser correlation spectroscopy after dilution with deionized water, instrument: Malvern Zetasizer 1000, Malver Inst. Limited.

The solids content of the polyurethane dispersions, which is preferably used to produce the cosmetic composition according to the invention, is generally 10 to 70% by weight, preferably 30 to 65% by weight, particularly preferably 40 to 60% by weight. The solids contents are ascertained in each case by heating a weighed sample to 125° C. to constant weight. At constant weight, the solids content is calculated by reweighing the sample.

Preferably, these polyurethane dispersions include less than 5% by weight, particularly preferably less than 0.2% by weight, based on the mass of the dispersions, of unbound organic amines. The content in the cosmetic composition according to the invention is accordingly even lower.

Suitable polyisocyanates of component A1) are in particular the aliphatic, aromatic or cycloaliphatic, preferably aliphatic or cycloaliphatic, polyisocyanates having an NCO functionality of greater than or equal to 2 that are known per se to those skilled in the art.

Examples of such suitable polyisocyanates are 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof of any isomer content, 1,4-cyclohexylene diisocyanate, 4-isocyanatomethyl-1,8-octane diisocyanate (nonane triisocyanate), 1,4-phenylene diisocyanate, 2,4- and/or 2,6-tolylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI) and alkyl 2,6-diisocyanatohexanoates (lysine diisocyanates) having C₁-C₈-alkyl groups.

The present invention therefore relates to the cosmetic composition according to the invention, wherein the at least one polyisocyanate is selected from the group consisting of 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof of any desired isomer content, 1,4-cyclohexylene diisocyanate, 4-isocyanatomethyl-1,8-octane diisocyanate (nonane triisocyanate), 1,4-phenylene diisocyanate, 2,4- and/or 2,6-tolylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI) and alkyl 2,6-diisocyanatohexanoates (lysine diisocyanates) having C₁-C₈-alkyl groups and mixtures thereof.

In addition to the aforementioned polyisocyanates, it is also possible to use modified diisocyanates having a functionality ≥2 and having uretidione, isocyanurate, urethane, allophanate, biuret, iminooxadiazinedione or oxadiazinetrione structure and also mixtures thereof also pro rata.

Preferably, no aromatic constituents are used in the cosmetic compositions according to the invention. Preference is given in accordance with the invention to polyisocyanates or polyisocyanate mixtures of the aforementioned type having exclusively aliphatically or cycloaliphatically bonded isocyanate groups or mixtures thereof and an average NCO functionality of the mixture of 2 to 4, preferably of 2 to 2.6 and particularly preferably of 2 to 2.4, especially preferably 2.

According to the invention, particular preference is given to using hexamethylene diisocyanate, isophorone diisocyanate or the isomeric bis(4,4′-isocyanatocyclohexyl)methane and mixtures of the aforementioned diisocyanates.

Used as A2) are polymeric polyols having a number-average molecular weight Mn of preferably 400 to 8000 g/mol, more preferably of 400 to 6000 g/mol and particularly preferably of 600 to 3000 g/mol. These preferably have an OH functionality of 1.5 to 6, more preferably of 1.8 to 3, most preferably of 1.9 to 2.1.

According to the invention, the expression “polymeric” polyols means in particular that the polyols mentioned have at least two, preferably at least three, repeat units linked to one another.

Such polymeric polyols are the polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols known per se in polyurethane coatings technology. These may be used in A2) individually or in any desired mixtures with one another.

The polyester polyols preferably used are, for example, the conventional polycondensates of di- and optionally tri- and tetraols and di- and optionally tri- and tetracarboxylic acids or hydroxycarboxylic acids or lactones. Also employable instead of the free polycarboxylic acids are the corresponding polycarboxylic anhydrides or corresponding polycarboxylic esters of lower alcohols for producing the polyesters.

Examples of suitable diols are ethylene glycol, butylene glycol, diethylene glycol, triethylene glycol, polyalkylene glycols such as polyethylene glycol and also 1,2-propanediol, 1,3-propanediol, 1,3-butanediol, 1,4-butanediol, 1,6-hexanediol and isomers, neopentyl glycol or neopentyl glycol hydroxypivalate, wherein 1,6-hexanediol and isomers, 1,4-butanediol, neopentyl glycol and neopentyl glycol hydroxypivalate are preferred. In addition, it is also possible to use polyols such as trimethylolpropane, glycerol, erythritol, pentaerythritol, trimethylolbenzene or trishydroxyethyl isocyanurate.

Employable dicarboxylic acids include phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. It is also possible according to the invention to use the corresponding anhydrides.

Provided that the average functionality of the polyol to be esterified is >2, it is additionally also possible to use monocarboxylic acids, such as benzoic acid and hexanecarboxylic acid, as well.

Preferred acids are aliphatic or aromatic acids of the type mentioned above. Adipic acid and/or succinic acid are particularly preferred.

Hydroxycarboxylic acids that may be co-used as reaction participants in the preparation of a polyester polyol having terminal hydroxyl groups are for example hydroxycaproic acid, hydroxybutyric acid, hydroxydecanoic acid, hydroxystearic acid and the like. Suitable lactones are caprolactone, butyrolactone and homologs. Preference is given to caprolactone.

According to the invention, particularly preferred as component A2) for the preparation of the polyurethanes are polyester polyols having a number-average molecular weight of 600 to 3000 g/mol, in particular aliphatic polyester polyols based on aliphatic carboxylic acids and aliphatic polyols, in particular based on adipic acid and/or succinic acid and aliphatic alcohols such as hexanediol, butanediol and/or neopentyl glycol.

It is likewise possible, as component A2), to use polycarbonates containing hydroxyl groups, preferably polycarbonate diols, having number-average molecular weights Mn of preferably 400 to 8000 g/mol, preferably 600 to 3000 g/mol. These are obtainable by reacting carbonic acid derivatives, such as diphenyl carbonate, dimethyl carbonate or phosgene, with polyols, preferably diols.

Examples of such diols are ethylene glycol, propane-1,2- and 1,3-diol, butane-1,3- and 1,4-diol, hexane-1,6-diol, octane-1,8-diol, neopentyl glycol, 1,4-bishydroxymethylcyclohexane, 2-methylpropane-1,3-diol, 2,2,4-trimethylpentane-1,3-diol, dipropylene glycol, polypropylene glycols, dibutylene glycol, polybutylene glycols, bisphenol A, and lactone-modified diols of the aforementioned type.

It is preferable when the diol component comprises 40% to 100% by weight hexanediol, preference being given to hexane-1,6-diol and/or hexanediol derivatives. Such hexanediol derivatives are based on hexanediol and have not only terminal OH groups but also ester groups or ether groups. Such derivatives are obtainable by reaction of hexanediol with excess caprolactone or by etherification of hexanediol with itself to afford di- or trihexylene glycol.

Instead of or in addition to pure polycarbonate diols, polyether-polycarbonate diols may also be used in A2).

Hydroxyl-containing polycarbonates are preferably linear.

Polyether polyols may likewise be used as component A2).

Particularly suitable examples are the polytetramethylene glycol polyethers known per se in polyurethane chemistry, as obtainable by cationic ring-opening polymerization of tetrahydrofuran.

Likewise suitable polyether polyols are the addition products, known per se, of styrene oxide, ethylene oxide, propylene oxide, butylene oxide and/or epichlorohydrin onto di- or polyfunctional starter molecules. Polyalkylene glycols in particular, such as polyethylene glycols, polypropylene glycols and/or polybutylene glycols, are employable, especially with the abovementioned preferred molecular weights.

Suitable starter molecules that may be used are all compounds known from the prior art, for example water, butyldiglycol, glycerol, diethylene glycol, trimethylolpropane, propylene glycol, sorbitol, ethylenediamine, triethanolamine, butane-1,4-diol.

Particularly preferred components in A2) are polytetramethylene glycol polyethers and polycarbonate polyols or mixtures thereof, polytetramethylene glycol polyethers being particularly preferred.

Accordingly, in preferred embodiments of the invention, component A2) is:
mixtures comprising at least one polyether polyol and at least one polycarbonate polyol,
mixtures comprising more than one polyether polyol, or a mixture of two or more polyether polyols having different molecular weights, which are in particular poly(tetramethylene glycol) polyether polyols,
mixtures comprising more than one polyether polyol and at least one polycarbonate polyol, and
particularly preferably polyester polyols having a number average molecular weight of 600 to 3000 g/mol, in particular aliphatic polyester polyols based on aliphatic carboxylic acids and aliphatic polyols, in particular based on adipic acid and aliphatic alcohols such as hexanediol and/or neopentyl glycol, in which component A) according to the definition essentially comprises neither ionic nor ionogenic groups.

As component A3) it is optionally possible to use polyols, especially non-polymeric polyols, of preferred said molecular weight range from 62 to 399 mol/g having up to 20 carbon atoms, such as ethylene glycol, diethylene glycol, triethylene glycol, propane-1,2-diol, propane-1,3-diol, butane-1,4-diol, 1,3-butylene glycol, cyclohexanediol, cyclohexane-1,4-dimethanol, hexane-1,6-diol, neopentyl glycol, hydroquinone dihydroxyethyl ether, bisphenol A (2,2-bis(4-hydroxyphenyl)propane), hydrogenated bisphenol A (2,2-bis(4-hydroxycyclohexyl)propane), trimethylolpropane, trimethylolethane, glycerol, pentaerythritol and any desired mixtures thereof with one another.

Also suitable are esterdiols of the cited molecular weight range such as alpha-hydroxybutyl epsilon-hydroxycaproate, omega-hydroxyhexyl gamma-hydroxybutyrate, beta-hydroxyethyl adipate or bis(beta-hydroxyethyl) terephthalate.

Monofunctional isocyanate-reactive compounds containing hydroxyl groups can also be used as component A3). Examples of such monofunctional compounds are ethanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

In a preferred embodiment of the invention, the polyurethane used according to the invention comprises less than about 10% by weight component A3), preferably less than 5% by weight component A3), based in each case on the total mass of the polyurethane, even more preferably component A3) is not used to produce the polyurethane.

One or more, in particular isocyanate reactive, non-ionic hydrophilizing agents, are optionally used as component A4) for the preparation of the polyurethanes used according to the invention. The hydrophilizing agents used as component A4) differ in particular from components A2) and A3).

Suitable non-ionically hydrophilizing compounds as component A4) are, for example, polyoxyalkylene ethers having isocyanate-reactive groups, such as hydroxyl, amino or thiol groups. Preference is given to monohydroxy-functional polyalkylene oxide polyether alcohols having a statistical average of 5 to 70, preferably 7 to 55, ethylene oxide units per molecule, as obtainable in a manner known per se by alkoxylation of suitable starter molecules (see, for example, Ullmanns Encyclopadie der technischen Chemie [Ullmann's Encyclopedia of Industrial Chemistry], 4th edition, volume 19, Verlag Chemie, Weinheim pp. 31 to 38). These are either pure polyethylene oxide ethers or mixed polyalkylene oxide ethers and they contain at least 30 mol %, preferably at least 40 mol %, based on all alkylene oxide units present, of ethylene oxide units.

Particularly preferred non-ionic compounds are monofunctional mixed polyalkylene oxide polyethers having 40 to 100 mol % of ethylene oxide units and 0 to 60 mol % of propylene oxide units.

Suitable starter molecules for such non-ionic hydrophilizing agents are especially saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or olein alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine, and heterocyclic secondary amines such as morpholine, pyrrolidine, piperidine or 1H-pyrazole. Preferred starter molecules are saturated monoalcohols of the abovementioned type. It is particularly preferable to use diethylene glycol monobutyl ethers or n-butanol as starter molecules.

Alkylene oxides suitable for the alkoxylation reaction are in particular ethylene oxide and propylene oxide, which can be used in the alkoxylation reaction in any desired sequence or else in a mixture.

Component B) is preferably selected from primary or secondary amines and/or diamines. It especially includes diamines.

As component B), in particular, amines may be used having no ionic or ionogenic groups, such as anionically hydrophilizing groups (component B1), and amines may be used having ionic or ionogenic groups, such as in particular anionically hydrophilizing groups (component B2). Preferably, in step B) of the reaction of the prepolymer, a mixture of component B1) and component B2) is reacted.

For example, components B1) used may be organic di- or polyamines such as for example 1,2-ethylenediamine, 1,2- and 1,3-diaminopropane, 1,4-diaminobutane, 1,6-diaminohexane, isophoronediamine (IPDA), an isomeric mixture of 2,2,4- and 2,4,4-trimethylhexamethylenediamine, 2-methylpentamethylenediamine, diethylenetriamine, 4,4-diaminodicyclohexylmethane, hydrazine hydrate and/or dimethylethylenediamine.

In addition, components B1) used may also be compounds that have not only a primary amino group but also secondary amino groups, or not only a primary or secondary amino group but also OH groups. Examples thereof are primary/secondary amines such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

Furthermore, components B1) used may also be monofunctional isocyanate-reactive amine compounds, for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide amines formed from diprimary amines and monocarboxylic acids, monoketime of diprimary amines, primary/tertiary amines, such as N,N-dimethylaminopropylamine.

Components B1) used are preferably ethylene-1,2-diamine, bis(4-aminocyclohexyl)methane, 1,4-diaminobutane, isophoronediamine, ethanolamine, diethanolamine and/or diethylenetriamine.

Component B) particularly preferably comprises at least one component B2). Suitable anionically hydrophilizing compounds as component B2 preferably comprise a sulfonic acid or sulfonate group, particularly preferably a sodium sulfonate group. Suitable anionically hydrophilizing compounds as component B2) are especially the alkali metal salts of mono- and diaminosulfonic acids. Examples of such anionic hydrophilizing agents are salts of 2-(2-aminoethylamino)ethanesulfonic acid, ethylenediaminepropylsulfonic or ethylenediaminebutylsulfonic acid, 1,2- or 1,3-propylendiamine-β-ethylsulfonic acid or taurine. The salt of cyclohexaminopropanesulfonic acid (CAPS) from WO-A 01/88006 can be used as an anionic hydrophilizing agent.

Particularly preferred anionic hydrophilizing agents B2) are those that comprise sulfonate groups as ionic groups and two amino groups, such as the salts of 2-(2-aminoethylamino)ethylsulfonic acid and propylene-1,3-diamine-β-ethylsulfonic acid.

The polyurethanes used according to the invention particularly preferably comprise at least one sulfonate group.

The anionic group in component B2) may optionally also be a carboxylate or carboxylic acid group. In that case, component B2) is preferably selected from diaminocarboxylic acids. However, this embodiment is less preferred since carboxylic acid-based components B2) have to be used in higher concentrations.

Mixtures of anionic hydrophilizing agents B2) and non-ionic hydrophilizing agents A4) may also be used for hydrophilization.

In a preferred embodiment for producing the specific polyurethane dispersions, components A1) to A4) and B1) to B2) are used in the following amounts, where the individual amounts always add up to 100% by weight:

5% to 40% by weight component A1),

55% to 90% by weight A2),

0.5 to 20% by weight of the sum total of components A3) and/or B1)

0.1% to 25% by weight of the sum total of components A4) and/or B2), with particular preference being given to using 0.1% to 5% by weight of anionic or potentially anionic hydrophilizing agents B2), based on the total amounts of components A1) to A4) and B1) to B2).

In a preferred embodiment for preparation of the specific polyurethane dispersions, components A1) to A4) and B1) to B2) are used in the following amounts, where the individual amounts always add up to 100% by weight:

5% to 35% by weight component A1),

60% to 90% by weight A2),

0.5 to 15% by weight of the sum total of components A3) and/or B1)

0.1% to 15% by weight of the sum total of components A4) and/or B2), with particular preference being given to using 0.2% to 4% by weight of anionic or potentially anionic hydrophilizing agents B2), based on the total amounts of components A1) to A4) and B1) to B2).

In a very particularly preferred embodiment for producing the specific polyurethane dispersions, the components A1) to A4) and B1) to B2) are used in the following amounts, wherein the individual amounts always add up to 100% by weight:

10% to 30% by weight component A1),

65% to 85% by weight A2),

0.5 to 14% by weight of the sum total of components A3) and/or B1)

0.1% to 13.5% by weight of the sum total of components A4) and/or B2), with particular preference being given to using 0.5% to 3.0% by weight of anionic or potentially anionic hydrophilizing agents of B2), based on the total amounts of components A1) to A4) and B1) to B2).

The production of the polyurethane dispersions may be carried out in one or more stages in homogeneous phase or, in the case of a multistage reaction, partially in disperse phase. Complete or partial performance of polyaddition from A1) to A4) is preferably followed by a dispersing, emulsifying or dissolving step. This is optionally followed by a further polyaddition or modification in disperse phase.

Any prior art method can be used, for example the prepolymer mixing method, the acetone method or the melt dispersing method. Preference is given to employing the acetone method.

For production by the acetone method, it is customary to form an initial charge including all or some of constituents A2) to A4) and the polyisocyanate component A1) for preparation of an isocyanate-functional polyurethane prepolymer, and optionally to dilute them with a solvent that is water-miscible but inert toward isocyanate groups, and heat them to temperatures in the range from 50° C. to 120° C. The isocyanate addition reaction can be accelerated using the catalysts known in polyurethane chemistry.

Suitable solvents are the customary aliphatic keto-functional solvents, such as acetone, 2-butanone, which can be added not just at the start of the preparation but optionally also in portions at a later stage. Acetone and 2-butanone are preferred and acetone is particularly preferred. The addition of other solvents without isocyanate-reactive groups is also possible, but not preferred.

Subsequently, any constituents of A1) to A4) not added at the start of the reaction are added.

In the production of the polyurethane prepolymer from A1) to A4), the amount of substance ratio of isocyanate groups to isocyanate-reactive groups is generally 1.05 to 3.5, preferably 1.1 to 3.0, particularly preferably 1.1 to 2.5.

Components A1) to A4) are converted partly or fully to the prepolymer, but preferably fully.

Polyurethane prepolymers containing free isocyanate groups are thus obtained in neat form or in solution.

In the neutralization step, partial or complete conversion of potentially anionic groups to anionic groups is accomplished using bases such as tertiary amines, e.g. trialkylamines having 1 to 12 and preferably 1 to 6 carbon atoms, particularly preferably 2 to 3 carbon atoms in each alkyl radical, or especially preferably alkali metal bases such as the corresponding hydroxides. The use of organic amines is not preferred.

Usable neutralizing agents are preferably inorganic bases, such as aqueous ammonia solution or sodium hydroxide or potassium hydroxide; preference is given to sodium hydroxide and potassium hydroxide.

The molar amount of the bases is between 50 and 125 mol %, preferably between 70 and 100 mol %, of the molar amount of the acid groups to be neutralized. The neutralization can also be effected simultaneously with the dispersing when the dispersion water already contains the neutralizing agent.

Subsequently, if not yet effected or only partially effected, the prepolymer obtained is dissolved using aliphatic ketones such as acetone or 2-butanone in a further process step.

Components A1) to A4) are converted partly or fully to the prepolymer, but preferably fully. Polyurethane prepolymers containing free isocyanate groups are thus obtained in neat form or in solution.

In the chain extension of stage B), NH₂— and/or NH-functional components are reacted with the still remaining isocyanate groups of the prepolymer. It is preferable when the chain extension/termination is carried out prior to the dispersing in water.

Suitable components B) for chain extension are particularly organic di- or polyamines B1) such as ethylenediamine, 1,2- and 1,3-diaminopropane, 1,4-diaminobutane, 1,6-diaminohexane, isophoronediamine, isomeric mixture of 2,2,4- and 2,4,4-trimethylhexamethylenediamine, 2-methylpentamethylenediamine, diethylenetriamine, diaminodicyclohexylmethane and/or dimethylethylendiamine.

Also employable are compounds B1) are compounds which comprise not only a primary amino group but also secondary amino groups or not only an amino group (primary or secondary) but also OH groups. Examples of these are primary/secondary amines such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine for chain extension or termination.

Chain termination is typically accomplished using amines B1) having an isocyanate-reactive group such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide amines formed from diprimary amines and monocarboxylic acids, monoketimes of diprimary amines, primary/tertiary amines, such as N,N-dimethylaminopropylamine.

When anionic hydrophilizing agents according to definition B2) having NH₂ groups or NH groups are used for chain extension, the chain extension of the prepolymers is preferably effected prior to the dispersing.

The degree of chain extension, i.e. the equivalents ratio of NCO-reactive groups of the compounds used for chain extension and chain termination to free NCO groups of the prepolymer, is generally between 40% and 150%, preferably between 50% and 110%, particularly preferably between 60% and 100%.

The aminic components B1) and B2) may optionally be used in water- or solvent-diluted form in the process of the invention, individually or in mixtures, any sequence of addition being possible in principle.

When water or organic solvents are included as a diluent, the diluent content in the component for chain extension used in B) is preferably in the range from 40% to 95% by weight.

The dispersing preferably follows the chain extension. To this end, the dissolved and chain-extended polyurethane polymer is either introduced into the dispersion water, optionally under high shear, for example vigorous stirring, or, conversely, the dispersion water is stirred into the chain-extended polyurethane polymer solutions. It is preferable when the water is added to the dissolved, chain-extended polyurethane polymer.

The solvent still present in the dispersions after the dispersion step is typically then removed by distillation. Removal even during the dispersing is likewise possible.

The residual content of organic solvents in the polyurethane dispersions thus produced is typically less than 10% by weight, preferably less than 3% by weight, based on the total dispersion.

The pH of the aqueous polyurethane dispersions used in accordance with the invention is typically less than 8.0, preferably less than 7.5, and is more preferably between 5.5 and 7.5.

The cosmetic composition according to the invention also comprises 0.1 to 7.5% by weight, based on the aqueous dispersion comprising the at least one polyurethane, of a mixture comprising at least one alkanediol. The cosmetic composition according to the invention preferably comprises 0.5 to 5.0% by weight, particularly preferably 1.0 to 3.0% by weight, based on the aqueous dispersion comprising the at least one polyurethane, of a mixture comprising at least one alkanediol.

According to the invention, an alkanediol is understood to mean an organic compound which, in addition to a linear or branched, saturated or unsaturated alkane radical having 2 to 12 carbon atoms, has at least two hydroxyl functions. In addition to the structural elements mentioned, the at least one alkanediol used according to the invention may also have other functional groups, preferably selected from the group consisting of ester, ketone, carboxylic acid, aldehyde group and combinations thereof. Furthermore, aromatic units, for example phenyl, benzyl, or naphthyl units, can also be bonded to or in the saturated or unsaturated alkane radical.

The mixture used according to the invention comprises at least one alkanediol. In particular, the mixture used according to the invention preferably comprises at least one alkanediol selected from the group consisting of 2-methyl-1,3-propanediol (CAS 2163-42-0), 1,2-octanediol (caprylyl glycol, CAS 1117-86-8), isomers thereof and mixtures thereof.

The present invention therefore preferably relates to the cosmetic composition according to the invention, wherein the at least one alkanediol is selected from the group consisting of 2-methyl-1,3-propanediol, 1,2-octanediol (caprylyl glycol), isomers thereof and mixtures thereof. Particularly preferably used according to the invention is a mixture comprising, preferably consisting of, 2-methyl-1,3-propanediol and 1,2-octanediol.

If a mixture comprising 2-methyl-1,3-propanediol and 1,2-octanediol, preferably consisting of 2-methyl-1,3-propanediol and 1,2-octanediol, is used in the composition according to the invention, these are preferably present in each case in an amount of 10 to 90% by weight, the sum total of the amounts of both components being 100% by weight in each case. 1,2-Octanediol is preferably present in an amount of 5 to 50% by weight, particularly preferably 10 to 40% by weight, especially preferably 20 to 35% by weight, based in each case on the mixture, and 2-methyl-1,3-propanediol is present in an amount of 50 to 95% by weight, particularly preferably 60 to 90% by weight, especially preferably 65 to 80% by weight, based in each case on the mixture, the sum total of the amounts of 2-methyl-1,3-propanediol and 1,2-octanediol being 100% by weight in each case.

According to the invention, other compounds may be present in the mixture used in addition to the at least one alkanediol. In addition to the at least one alkanediol, preferably present in the mixture is at least one further organic compound, for example selected from the group consisting of aliphatic, araliphatic or aromatic alcohols, carboxylic acids and mixtures thereof, for example benzoic acid, phenoxyethanol and/or 3-phenylpropanol and/or isomers thereof. The further compound present in addition to the at least one alkanediol is preferably selected from the group consisting of 3-phenylpropanol, isomers thereof and mixtures thereof.

The present invention particularly preferably relates to the cosmetic composition according to the invention comprising at least one polyurethane and, based on the at least one polyurethane, 0.1 to 7.5% by weight of at least one mixture, wherein the mixture comprises 2-methyl-1,3-propanediol, 1,2-octanediol (caprylyl glycol) and 3-phenylpropanol, preferably consists of these three components.

The present invention particularly preferably relates to the cosmetic composition according to the invention, wherein the mixture comprises, preferably consists of, 70 to 89% by weight, preferably 76 to 87% by weight 2-methyl-1,3-propanediol, 10 to 30% by weight, preferably 10 to 20% by weight 1,2-octanediol (caprylyl glycol) and 1 to 5% by weight, preferably 3 to 4% by weight 3-phenylpropanol, the sum total of the components mentioned being 100% by weight.

In addition to the at least one polyurethane and the mixture described above, the cosmetic composition according to the invention comprises further ingredients, in particular at least one cosmetically and/or therapeutically active ingredient. The further ingredients present according to the invention can be different depending on the respective intended use of the cosmetic composition, i.e. for cleansing and/or care of the skin, as sun protection, as decorative cosmetic composition or as hair setting agent.

The at least one cosmetically and/or therapeutically active ingredient is generally present in the cosmetic composition according to the invention in amounts known to those skilled in the art.

In the context of the present invention, the cosmetic composition according to the invention may advantageously be a cream, lotion, milk, gel, oil, balm, aqueous solution, peel-off masks, spray, aerosol, masks and all make-up forms known to the person skilled in the art.

The at least one polyurethane in the cosmetic composition according to the invention is generally present to an extent of 0.5 to 45% by weight, preferably 10 to 45% by weight, based in each case on the total cosmetic composition.

The composition according to the invention, which comprises the polyurethane described above or an aqueous dispersion thereof, is intended in particular to fulfil the aforementioned properties of a cosmetic product, in particular skin care or sun protection product.

In a preferred embodiment of the present invention, the cosmetic composition according to the invention remains at least partially on the skin and/or hair, especially facial skin, after application. After application of the composition according to the invention to the skin and/or hair and/or nails, in particular facial skin, a film is preferably formed which is left on the skin and/or hair, in particular facial skin, for a certain time, for example 1 to 60 minutes, and is then optionally removed. In a further embodiment, the cosmetic composition according to the invention may also remain on the skin, in particular the facial skin, overnight. If the composition according to the invention is applied to nails, it can be removed after a certain time, for example several days, preferably as a whole.

In the context of the present invention, the cosmetic compositions differ in particular according to their consistency: cream (viscous), lotion and milk (flowable), gels (semi-solid), oils and balms and aqueous solutions (liquid). Depending on their structure, the compositions according to the invention may be used, for example, as face creams, day or night creams, body lotions, peel-off masks, etc. It is optionally possible for the compositions according to the invention to be used as a pharmaceutically active product or to contain pharmaceutically active ingredients.

The cosmetic compositions according to the invention may be, for example, in the form of oil-in-water, silicone-in-water, water-in-oil, water-in-silicone, oil-in-water-in-oil, water-in oil-in-water emulsion.

The cosmetic compositions according to the invention can also be foamed with a propellant gas. The emulsions described above can be stabilized by one or more 0/W, W/0 or W/Si emulsifiers, thickeners (such as hydrodispersion) or solids (such as a Pickering emulsion).

The cosmetic compositions may comprise one or more emulsifiers or surface-active agents.

Thus, oil-in-water emulsions (0/W) in particular according to the invention preferably comprise at least one emulsifier having an HLB value >7 and optionally a co-emulsifier.

O/W emulsifiers may advantageously be selected from the group of the non-ionic, anionic, cationic or amphoteric emulsifiers.

Among the non-ionic emulsifiers are:

a) partial fatty acid esters and fatty acid esters of polyhydric alcohols and ethoxylated derivatives thereof

b) ethoxylated fatty alcohols and fatty acids.

c) ethoxylated fatty amines, fatty acid amides, fatty acid alkanolamides

d) alkylphenol polyglycol ethers, e) ethoxylated fatty alcohol ethers.

Particularly advantageous non-ionic O/W emulsifiers are ethoxylated fatty alcohols or fatty acids, preferably PEG-100 stearate, PEG-40 stearate, PEG-50 stearate, ceteareth-20, ceteth-20, steareth-20, ceteareth-12, ceteth-12, steareth-12, esters of mono-, oligo- or polysaccharides with fatty acids, preferably cetearyl glucoside, methyl glucose distearate, glyceryl monostearates (self-emulsifying), sorbitan esters such as sorbitan stearates (Tween® 20 and Tween® 60 from Uniqema), sorbitan palmitates (Span® 40, Uniqema), glyceryl stearyl citrate, sucrose esters such as sucrose stearate, PEG-20 methylglucose sesquistearate, dicarboxylic acid esters of fatty alcohol such as dimyristyl tartrate.

Advantageous anionic emulsifiers are soaps, e.g. sodium or triethanolamine salts of stearic or palmitic acid, esters of citric acid such as glyceryl stearate citrate, fatty alcohol sulfates and mono-, di- and trialkylphosphoric acid esters and ethoxylates thereof.

Among the cationic emulsifiers are quaternary ammonium compounds having a long-chain aliphatic radical, e.g. distearyldimonium chloride.

Among the amphoteric emulsifiers are:

a) alkylaminoalkanoic acids

b) betaines, sulfobetaines

c) imidazoline derivatives.

There are also naturally occurring emulsifiers which include beeswax, wool wax, lecithin and sterols.

Suitable coemulsifiers used for O/W emulsions according to the invention may be fatty alcohols having 8 to 30 carbon atoms, monoglyceryl esters of saturated or unsaturated, branched or unbranched alkanecarboxylic acids having a chain length of 8 to 24 carbon atoms, especially 12 to 18 carbon atoms, propylene glycol esters of saturated or unsaturated, branched or unbranched alkanecarboxylic acids having a chain length of 8 to 24 carbon atoms, especially 12 to 18 carbon atoms, and sorbitan esters of saturated or unsaturated, branched or unbranched alkanecarboxylic acids having a chain length of 8 to 24 carbon atoms, especially 12 to 18 carbon atoms.

Particularly advantageous co-emulsifiers are glyceryl monostearate, glyceryl monooleate, diglyceryl monostearate, sorbitan monoisostearate, sucrose distearate, cetyl alcohol, stearyl alcohol, behenyl alcohol, isobehenyl alcohol and polyethylene glycol(2) stearyl ether (steareth-2).

It may be advantageous in the context of the present invention to use further emulsifiers. For example, the water resistance of the preparations according to the invention can be increased. Examples of suitable emulsifiers are, for example, alkyl methicone copolyols and alkyl dimethicone copolyols, in particular cetyl dimethicone copolyol, lauryl methicone copolyol, W/0 emulsifiers such as sorbitan stearate, glyceryl stearate, glycerol stearate, sorbitan oleate, lecithin, glyceryl isostearate, polyglyceryl-3 oleate, polyglyceryl-3 diisostearate, PEG-7 hydrogenated castor oil, polyglyceryl-4 isostearate, acrylate/C10-30-alkyl acrylate crosspolymer, sorbitan isostearate, poloxamer 101, polyglyceryl-2 dipolyhydroxystearate, polyglyceryl-3 diisostearate, polyglyceryl-4 dipolyhydroxystearate, PEG-30 dipolyhydroxystearate, diisostearoylpolyglyceryl-3 diisostearate, glycol distearate, and polyglyceryl-3 dipolyhydroxystearate.

The cosmetic compositions according to the invention, in particular the 0/W compositions, may advantageously comprise thickeners for the water phase. Advantageous thickeners are:
crosslinked or non-crosslinked acrylic or methacrylic acid homo- or copolymers. These include crosslinked homopolymers of methacrylic acid or acrylic acid, copolymers of acrylic acid and/or methacrylic acid and monomers derived from other acrylic or vinyl monomers, such as C10-30 alkyl acrylates, C10-30 alkyl methacrylates, vinyl acetate and vinylpyrrolidone.
thickening polymers of natural origin, for example based on cellulose, guar gum, xanthan, scleroglucan, gellan gum, rhamsan and karaya gum, alginates, maltodextrin, starch and its derivatives, locust bean gum, hyaluronic acid, carrageenan.
non-ionic, anionic, cationic or amphoteric associative polymers, for example based on polyethylene glycols and derivatives thereof, or polyurethanes.
crosslinked or non-crosslinked homopolymers or copolymers based on acrylamide or methacrylamide, such as homopolymers of 2-acrylamido-2-methylpropanesulfonic acid, copolymers of acrylamide or methacrylamide and methacryloyloxyethyltrimethylammonium chloride or copolymers of acrylamide and 2-acrylamido-2-methylpropanesulfonic acid.

Particularly advantageous thickeners are thickening polymers of natural origin, crosslinked acrylic acid or methacrylic acid homo- or copolymers, and crosslinked copolymers of 2-acrylamido-2-methylpropanesulfonic acid.

Very particularly advantageous thickeners are xanthan gum, such as the products supplied under the Keltrol® and Kelza® names by CP Kelco or the products from RHODIA with the Rhodopol name and guar gum, such as those sold under the Jaguar® HP105 name by RHODIA.

Very particularly advantageous thickeners are crosslinked homopolymers of methacrylic acid or acrylic acid, which are available from Lubrizol under the names Carbopol® 940, Carbopol® 941, Carbopol® 980, Carbopol® 981, Carbopol® ETD 2001, Carbopol® EDT 2050, Carbopol® 2984, Carbopol® 5984 and Carbopol® Ultrez 10, from the company 3V, which are commercially available under the names Synthalen® K, Synthalen® L and Synthalen® MS.

Very particular advantageous thickeners are crosslinked copolymer of acrylic acid or methacrylic acid and a C10-30-alkyl acrylate or C10-30-alkyl methacrylate and copolymers of acrylic acid or methacrylic acid and vinylpyrrolidone. Such copolymers are commercially available, for example, from Lubrizol under the names Carbopol® 1342, Carbopol® 1382, Pemulen® TR1 or Pemulen® TR2 and from Ashland under the names Ultrathix® P-100 (INCI: Acrylic Acid/VP Crosspolymer).

Very particularly advantageous thickeners are crosslinked copolymers of 2-acrylamido methylpropanesulfonic acid. Such copolymers are obtainable, for example, from Clariant under the names Aristoflex® AVC (INCI: Ammonium Acryloyldimethyltaurate/VP Copolymer).

These thickeners are generally present at a concentration of about 0% to 2% by weight, preferably 0% to 1% by weight, based on the total weight of the cosmetic composition according to the invention.

Further compositions according to the invention can be water-in-oil or water-in-silicone emulsions. Preference is given to water-in-oil (W/O) or water-in-silicone emulsions (W/Si) comprising one or more silicone emulsifiers (W/S) having an HLB value ≤8 or one or more W/O-emulsifiers having an HLB value <7 and optionally one or more O/W emulsifiers having an HLB value >10.

The silicone emulsifiers may advantageously be selected from the group comprising alkyl dimethicone copolyols, such as cetyl PEG/PPG 10/1 dimethicone copolyol (ABIL® EM 90 from Evonik) or lauryl PEG/PPG-18/18 dimethicone (Dow Corning® 5200 from Dow Corning Ltd.), and dimethicone copolyols such as PEG-10 dimethicone (KF-6017 from Shin Etsu), PEG/PPG-18/18 dimethicone (Dow Corning 5225C from Dow Corning Ltd.) or PEG/PPG-19/19 dimethicone (Dow Corning BY-11 030 from Dow Corning Ltd.) or trimethylsilylamodimethicone.

The W/0 emulsifiers having an HLB value <7 can advantageously be selected from the following group: fatty alcohols having 8 to 30 carbon atoms, monoglycerol esters of saturated and/or unsaturated, branched and/or unbranched alkanecarboxylic acids having a chain length of 8 to 24, in particular 12-18 carbon atoms, diglycerol esters of saturated and/or unsaturated, branched and/or unbranched alkanecarboxylic acids of a chain length from 8 to 24, in particular 12-18 carbon atoms, monoglycerol ethers of saturated and/or unsaturated, branched and/or unbranched alcohols of a chain length of 8 to 24, in particular 12-18 carbon atoms, diglycerol ethers of saturated and/or unsaturated, branched and/or unbranched alcohols of a chain length of 8 to 24, in particular 12-18 carbon atoms, propylene glycol esters of saturated and/or unsaturated, branched and/or unbranched alkanecarboxylic acids of a chain length of 8 to 24, in particular 12-18 carbon atoms, and sorbitan esters of saturated and/or unsaturated, branched and/or unbranched alkanecarboxylic acids of a chain length from 8 to 24, in particular 12-18 carbon atoms.

Particularly advantageous W/0 emulsifiers are: glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate and glyceryl monocaprylate.

Other possible W/O emulsifiers are selected from the group of compounds comprising polyglyceryl-2 dipolyhydroxystearate, PEG-30 dipolyhydroxystearate, cetyl dimethicone copolyol and poly glyceryl-3 diisostearate.

O/W emulsifiers having an HLB value >10 may advantageously be selected from the group comprising lecithin, trilaureth-4 phosphate, polysorbate 20, polysorbate 60, PEG-22-dodecyl glycol copolymer, sucrose stearate, and sucrose laurate.

For stabilization of the W/O emulsion according to the invention against sedimentation or flocculation of water droplets, an oil thickener may advantageously be used.

Particularly advantageous oil thickeners are organomodified clays such as organomodified bentonites (Bentone® 34 from Elementis), organomodified hectorite (Benton® 27 and Benton® 38 from Elementis) or organomodified montmorillonite, hydrophobic fumed silica, in which the silanol groups are substituted with trimethylsiloxy groups (AEROSIL® R812 from Evonik) or with dimethylsiloxy groups or polydimethylsiloxane (AEROSIL® R972, AEROSIL® R974 from Evonik, CAB-O-SIL® TS-610, CAB-O-SIL® TS-720 from Cabot), magnesium or aluminum stearate, or styrene copolymers such as styrene-butadiene-styrene, styrene-isopropene-styrene, styrene-ethylene/butene-styrene or styrene-ethylene/propene-styrene.

The thickener for the oil phase may be present in an amount of 0.1% to 5% by weight, based on the total weight of the emulsion, and preferably 0.4% to 3% by weight.

The aqueous phase may additionally comprise stabilizing agents. The stabilizing agent may be, for example, sodium chloride, magnesium chloride or magnesium sulfate, and mixtures thereof. Oils may be used in W/O, W/Si, and O/W emulsions.

If present, the fatty phase of the composition according to the invention may comprise a non-volatile oil and/or volatile oils and waxes. The 0/W composition advantageously comprises 0.01% to 45% by weight of oils, based on the total weight of the composition, and particularly advantageously 0.01% to 20% by weight of oils. The W/0 or W/Si composition advantageously comprises at least 20% by weight of oils, based on the total weight of the composition.

The non-volatile oil is advantageously chosen from the group consisting of mineral, animal, plant or synthetic origin, polar or non-polar oils, and mixtures thereof.

The lipid phase of the cosmetic composition according to the invention may advantageously be selected from the following group of substances:

mineral oils, mineral waxes, polar oils such as triglycerides of capric or caprylic acid, and also natural oils such as castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alkyl benzoates; silicone oils such as dimethylpolysiloxane s, diethylpoly siloxanes, diphenylpolysiloxanes and mixed forms thereof.

The polar oils are advantageously chosen from the group of:

a) esters of saturated and unsaturated, branched and/or unbranched alkanecarboxylic acids of chain length from 3 to 30 carbon atoms and saturated and/or unsaturated, branched and/or unbranched alcohols of chain length from 3 to 30 carbon atoms,

b) esters of aromatic carboxylic acids and saturated and/or unsaturated, branched and/or branched alcohols of chain length from 3 to 30 carbon atoms.

Such ester oils can then advantageously be chosen from the group of:

isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, isotridecyl isononanoate, 2-ethylhexyl palmitate, 2-ethylhexyl laurate, 2-ethylhexyl isostearate, 2-hexyldecyl stearate, 2-octyldodecyl palmitate, 2-ethylhexyl cocoate, oleyl oleate, oleyl erucate, erucyl oleate, erucyl erucate, dicaprylyl carbonate (Cetiol® CC) and cocoglycerides (Myritol® 331), and also synthetic, semi-synthetic and natural mixtures of such esters, for example jojoba oil.

c) alkyl benzoates, C12-15-alkyl benzoate (Finsolv® TN from Innospec Performance Chemicals) or 2-phenylethyl benzoate (X-Tend® 226 from Ashland)

d) lecithins and the fatty acid triglycerides, namely the triglyceryl esters of saturated and unsaturated, branched and/or unbranched alkanecarboxylic acids of chain length 8 to 24, especially 12 to 18, carbon atoms. For example, the fatty acid triglycerides may be selected from the group consisting of cocoglyceride, olive oil, sunflower oil, soybean oil, groundnut oil, rapeseed oil, almond oil, palm oil, coconut oil, castor oil, wheatgerm oil, grapeseed oil, safflower oil, evening primrose oil, macadamia nut oil, apricot kernel oil, avocado oil, and the like.

e) the dialkyl ethers and dialkyl carbonates, advantageous examples being dicaprylyl ether (Cetiol® OE from BASF) and/or dicaprylyl carbonate (for example Cetiol® CC from BASF).

f) saturated or unsaturated, branched or unbranched alcohols, such as octyldodecanol.

The non-volatile oil may likewise advantageously also be a non-polar oil chosen from the group consisting of branched and unbranched hydrocarbons, in particular mineral oil, vaseline oil, paraffin oil, squalane and squalene, polyolefins, for example polydecenes, hydrogenated polyisobutenes, C13-16 isoparaffin, and isohexadecane.

The non-polar non-volatile oil may be selected from the non-volatile silicone oils.

The non-volatile silicone oils may include the polydimethylsiloxanes (PDMS) that are optionally phenylated, such as phenyltrimethicone, or are optionally substituted by aliphatic and/or aromatic groups or by functional groups, for example hydroxyl groups, thiol groups and or amino groups;

polysiloxanes modified with fatty acids, fatty alcohols or polyoxyalkylenes, and mixtures thereof.

Particularly advantageous oils are 2-ethylhexyl isostearate, octyldodecanol, isotridecyl isononanoate, isoeicosane, 2-ethylhexyl cocoate, C12-15 alkyl benzoate, Caprylic/Capric Triglyceride, Dicaprylyl Ether, Mineral Oil, Dicaprylyl Carbonate, Cocoglyceride, Butylene Glycol Dicaprylate/Dicaprate,

Hydrogenated Polyisobutene, Cetaryl Isononanoate, Isodecyl Neopentanoate, Squalane, C13-16 Isoparaffin.

The composition according to the invention may further comprise a wax. In the context of the present document, a wax is defined as a lipophilic fatty substance that is solid at room temperature (25° C.) and shows a reversible solid/liquid change of state at a melting temperature between 30° C. and 200° C. Above the melting point, the viscosity of the wax is low and it becomes miscible with oils.

The wax is advantageously selected from the groups of natural waxes, for example cotton wax, carnauba wax, candelilla wax, esparto wax, japan wax, montan wax, sugarcane wax, beeswax, wool wax, shellac, microwaxes, ceresin, ozokerite, ouricury wax, cork fiber wax, lignite waxes, berry wax, shea butter, or synthetic waxes such as paraffin waxes, polyethylene waxes, waxes produced by Fischer-Tropsch synthesis, hydrogenated oils, fatty acid esters and glycerides that are solid at 25° C., silicone waxes and derivatives (alkyl derivatives, alkoxy derivatives and/or esters of polymethylsiloxane) and mixtures thereof. The waxes can be in the form of stable dispersions of colloidal wax particles which can be produced by known processes, for example according to “Microemulsions Theory and Practice”, L.M. Prince Ed., Academic Press (1977), pages 21 to 32.

The waxes may be present in amounts of 0% to 10% by weight, based on the total weight of the composition, and preferably 0% to 5% by weight.

The composition of the invention may further comprise a volatile oil selected from the group consisting of volatile hydrocarbons, siliconized oils, and fluorinated oils.

The volatile oil may be present in an amount of 0% to 25% by weight, based on the total weight of the emulsion, preferably 0% to 20% by weight, and more preferably 0% to 15% by weight.

In the context of the present document, a volatile oil is an oil which evaporates within less than one hour on contact with the skin at room temperature and atmospheric pressure. The volatile oil is liquid at room temperature and, at room temperature and atmospheric pressure, has a vapor pressure of 0.13 to 40,000 Pa (103 to 300 mm Hg), preferably 1.3 to 13,000 Pa (0.01 to 100 mm Hg), and particularly preferably 1.3 to 1300 Pa (0.01 to 10 mm Hg), and a boiling point of 150 to 260° C. and preferably 170 to 250° C.

A hydrocarbon oil is understood to mean a oil which is formed essentially from carbon atoms and hydrogen atoms, with or without oxygen atoms or nitrogen atoms, and does not contain any silicon atoms or fluorine atoms, and it may also consist of carbon atoms and hydrogen atoms; it may contain ester groups, ether groups, amino groups or amide groups.

A siliconized oil is understood to mean an oil containing at least one silicon atom and especially Si—O groups.

A fluorinated oil is understood as meaning an oil containing at least one fluorine atom.

The volatile hydrocarbon oil according to the invention may be selected from the hydrocarbon oils having a flash point of 40 to 102° C., preferably 40 to 55° C., and more preferably 40 to 50° C. For example, the volatile hydrocarbon oils are volatile hydrocarbon oils having 8 to 16 carbon atoms and mixtures thereof, in particular branched C₈-C₁₆-alkanes such as isoalkanes (also referred to as isoparaffins) having 8 to 16 carbon atoms, isododecane, isodecane, isohexadecane and, for example, the oils marketed under the trade names Isopars® or Permetyls®; and the branched C₈-C₁₆-esters, such as isohexyl neopentanoate and mixtures thereof.

Particularly advantageous are volatile hydrocarbon oils such as isododecane, isodecane, and isohexadecane.

The siliconized oil which is volatile in accordance with the invention may be selected from the siliconized oils having a flashpoint of 40 to 102° C., preferably a flashpoint exceeding 55° C. and not more than 95° C. and particularly preferably in the range from 65 to 95° C.

For example, the volatile siliconized oils are straight-chain or cyclic silicone oils having 2 to 7 silicon atoms, these silicones optionally containing alkyl or alkoxy groups having 1 to 10 carbon atoms.

Particularly advantageous are volatile siliconized oils such as octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

The volatile fluorinated oil does not generally have a flash point.

Examples of volatile fluorinated oils are nonafluoroethoxybutane, nonafluoromethoxybutane, decafluoropentane, tetradecafluorohexane, dodecafluoropentane, and mixtures thereof.

The cosmetically acceptable medium of the composition according to the invention comprises water and optionally a cosmetically suitable organic solvent miscible in water.

The water used in the composition according to the invention may be a blossom water, pure demineralized water, mineral water, thermal water, and/or seawater.

In the case of an O/W composition, the water content may be in the range from 40% to 95% by weight, preferably in the range from 50% to 90% by weight, most preferably in the range from 60% to 80% by weight, based on the total weight of the composition. In the case of a W/0 composition, the water content is in the range from 0% to 60% by weight, preferably in the range from 10% to 50% by weight, most preferably in the range from 30% to 50% by weight, based on the total weight of the composition.

The preferred solvents are selected, for example, from the aliphatic alcohols having C1-4 carbon atoms, such as ethanol and isopropanol; polyol and derivatives thereof such as propylene glycol, dipropylene glycol, butylene 1,3-glycol, polypropylene glycol, glycol ethers such as alkyl (C1-4) ethers of mono-, di- or tripropylene glycol or mono-, di- or triethylene glycol, and mixtures thereof.

The proportion of the solvent or solvents in the composition of the invention may, for example, be in the range from 0% to 25% by weight and preferably 0% to 10% by weight, based on the total weight of the composition.

The composition according to the invention may additionally comprise additives that are customary in cosmetics, such as antioxidants, light stabilizers and/or other auxiliaries and additives such as emulsifiers, surface-active substances, defoamers, thickeners, surfactants, active ingredients, moisturizers, fillers, UV filters, film formers, solvents, coalescing agents, flavorings, odor absorbers, perfumes, gel formers and/or other polymer dispersions such as dispersions based on polyacrylates, pigments, dyes, leveling agents and/or thixotropic agents, emollients, plasticizers, preservatives. The amounts of the various additives are known to those skilled in the art for the range to be used and, if present, range, for example, from 0.1% to 25% by weight based on the total weight of the composition.

The cosmetic composition according to the invention may also comprise sensory additives. Sensory additives are understood to mean colorless or white, mineral or synthetic, lamellar, spherical or elongated inert particles or a non-particulate sensory additive which, for example, further improve the sensory properties of the formulations and, for example, leave the skin feeling velvety or silky.

The sensory additives, if present, may be present in the composition according to the invention in an amount from 0.1% to 10% by weight and preferably from 0.1% to 7%, based in each case on the total weight of the composition.

Advantageous particulate sensory additives in the context of the present invention are talc, mica, silicon oxide, kaolin, starch and derivatives thereof, for example tapioca starch, di-starch phosphate, aluminium starch or sodium starch octenylsuccinate and the like, fumed silica, pigments having neither principally UV filter action nor colouring action, for example boron nitride, calcium carbonate, dicalcium phosphate, magnesium carbonate, magnesium hydrogencarbonate, hydroxyapatites, microcrystalline cellulose, powders of synthetic polymers such as polyamides, for example the polymers available under the “Nylon®” trade name, polyethylene, poly-β-alanine, polytetrafluoroethylene (“Teflon®”), polyacrylate, polyurethane, lauroylly sines, silicone resin, for example the polymers available under the “Tospearl®” trade name from Kobo Products Inc., hollow particles of polyvinylidene/acrylonitriles (Expancel® from Nouryon) or hollow particles of silicon oxide (Silica Beads® from MAPRECOS).

Advantageous non-particulate sensory additives may be selected from the group of dimethiconols, for example Dow Corning 1503 Fluid from Dow Corning Ltd., silicone copolymers, for example divinyldimethicone/dimethicone copolymer, Dow Corning HMW 2220 from Dow Corning Ltd., or silicone elastomers, for example dimethicone crosspolymer, Dow Corning 9040 Silicone Elastomer Blend from Dow Corning Ltd.

The composition according to the invention, in particular when used as a sun-protective composition, may comprise sunscreens, the total amount of sunscreens, if present, being from 0.1% by weight to 30% by weight, advantageously from 0.1% by weight to 20% by weight % by weight, particularly advantageously from 0.1% by weight to 10% by weight, based on the total weight of the composition according to the invention.

Sunscreen filters (also called UV filters) may be selected from organic filters, physical filters and mixtures thereof.

The composition according to the invention may comprise UV-A filters, UV-B filters or broad spectrum filters. The UV filters used may be oil-soluble or water-soluble. The attached list of UV filters mentioned below is of course not limiting.

Examples of UV-B filters include:
(1) salicylic acid derivatives, particularly homomenthyl salicylate, octyl salicylate and 4-isopropylbenzyl salicylate;
(2) cinnamic acid derivatives, particularly 2-ethylhexyl p-methoxycinnamate, available from Givaudan under the Parsol MCX® name, and isopentyl 4-methoxycinnamate;
(3) liquid β,β′-diphenylacrylate derivatives, particularly 2-ethylhexyl α,β′-diphenylacrylate or octocrylene, available from BASF under the UVINUL N539® name;
(4) p-aminobenzoic acid derivatives, particularly 2-ethylhexyl 4-(dimethylamino)benzoate, amyl 4-(dimethylamino)benzoate;
(5) 3-benzylidene camphor derivatives, particularly 3-(4-methylbenzylidene)camphor, commercially available from Merck under the EUSOLEX 6300® name, 3-benzylidenecamphor, benzylidenecamphorsulfonic acid and polyacrylamidomethylbenzylidenecamphor;
(6) 2-phenylbenzimidazole-5-sulfonic acid available under the EUSOLEX 232® name from Merck;
(7) 1,3,5-triazine derivatives, in particular: 2,4,6-tris[p-(2′-ethylhexyl-1′-oxycarbonyl)anilino]-1,3,5-triazine, supplied by BASF under the UVINUL T150® name, and dioctylbutamidotriazone, supplied by Sigma 3V under the UVASORB HEB® name;
(8) esters of benzalmalonic acid, particularly di(2-ethylhexyl) 4-methoxybenzalmalonate and 3-(4-(2,2-bisethoxycarbonylvinyl)phenoxy)propenyl)methoxysiloxane/dimethylsiloxane copolymer, available from DSM under the Parsol® SLX name; and
(9) mixtures of these filters.

Examples of UV-A filters include:
(1) dibenzoylmethane derivatives, particularly 4-(t-butyl)-4′-methoxydibenzoylmethane, which is supplied by Givaudan under the PARSOL 1789® name, and 1-phenyl-3-(4′-isopropylphenyl)propane-1,3-dione;
(2) benzene-1,4-[di(3-methylidenecamphor-10-sulfonic acid)], optionally fully or partly neutralized, commercially available under the MEXORYL SX® name from Chimex.
(3) hexyl 2-(4′-diethylamino-2′-hydroxybenzoyl)benzoate (also aminobenzophenone);
(4) silane derivatives or polyorganosiloxanes having benzophenone groups;
(5) anthranilates, particularly menthyl anthranilate, which is supplied by Symrise under the NEO HELIOPAN MA® name;
(6) compounds comprising at least two benzazolyl groups or at least one benzodiazolyl group per molecule, especially 1,4-bis(benzimidazolyl)phenylene-3,3′,5,5′-tetrasulfonic acid and salts thereof, available from Symrise;
(7) silicon derivatives of benzimidazolylbenzazoles that are N-substituted, or of benzofuranylbenzazoles, especially: 2-[1-[3-[1,3,3,3-tetramethyl-1-[(trimethylsilyl)oxy]disiloxanyl]propyl]-1H-benz imidazol-2-yl]benzoxazole; 2-[1-[3-[1,3,3,3-tetramethyl-1-[(trimethylsilyl)oxy]disiloxanyl]propyl]-1H-benz imidazol-2-yl]benzothiazole; 2-[1-(3-trimethylsilanylpropyl)-1H-benzimidazol-2-yl]benzoxazole; 6-methoxy-1,1′-bis(3-trimethylsilanylpropyl)-1H,1′H-[2,2′]dibenzimidazolylbenzoxazole; 2-[1-(3-trimethylsilanylpropyl)-1H-benzimidazol-2-yl]benzothiazole; which are described in patent application EP-A-1 028 120;
(8) triazine derivatives, especially 2,4-bis[5-1(dimethylpropyl)benzoxazol-2-yl-(4-phenyl)imino]-6-(2-ethylhexyl)imino-1,3,5-triazine, supplied by 3V under the Uvasorb®K2A name; and (9) mixtures thereof.

Examples of broadband filters are:
(1) benzophenone derivatives, for example 2,4-dihydroxybenzophenone (benzophenone-1); 2,2′,4,4′-tetrahydroxybenzophenone (benzophenone-2); 2-hydroxy-4-methoxybenzophenone (benzophenone-3) available from BASF under the name UNIVNUL M40®; 2-hydroxy methoxybenzophenone-5-sulfonic acid (benzophenone-4) and the sulfonate form thereof (benzophenone-5) available from BASF under the name UVINUL MS40®; 2,2′-dihydroxy-4,4′-dimethoxybenzophenone (benzophenone-6); 5-chloro-2-hydroxybenzophenone (benzophenone-7); 2,2′-dihydroxy-4-methoxybenzophenone (benzophenone-8); the disodium salt of 2,2′-dihydroxy-4,4′-dimethoxybenzophenone-5,5′-disulfonic acid (benzophenone-9); 2-hydroxy-4-methoxy-4′-methylbenzophenone (benzophenone-10); benzophenone-11; 2-hydroxy-4-(octyloxy)benzophenone (benzophenone-12).
(2) triazine derivatives, especially 2,4-bis{[4-2-ethylhexyloxy)-2-hydroxy]phenyl}-6-(4-methoxyphenyl)-1,3,5-triazine, which is supplied by BASF under the TINOSORB S® name, and 2,2′-methylenebis[6-(2H-benzotriazol-2-yl)-4-(1,1,3,3-tetramethylbutyl)phenol], available from BASF under the TINOSORB M® name; and
(3) 2-(1H-benzotriazol-2-yl)-4-methyl-6-[2-methyl-3-[1,3,3,3-tetramethyl-1-[(trimethylsilyl)oxy]disiloxanyl]propyl]phenol with the INCI name Drometrizole Trisiloxane.

It is also possible according to the invention to use a mixture of two or more filters and a mixture of UV-B filters, UV-A filters and broadband filters, and also mixtures with physical filters.

The physical filters may include the sulfates of barium, and oxides of titanium, particularly titanium dioxide, amorphous or crystalline in the form of rutile and/or anatase, of zinc, of iron, of zirconium, of cerium, of silicon, of manganese or mixtures thereof. The metal oxides may be in particle form with a size in the micrometer range or nanometer range (nanopigments). The mean particle sizes for the nanopigments are, for example, 5 to 100 nm.

The cosmetic composition according to the invention may also comprise moisturizers.

Particularly advantageous moisturizers in the context of the present invention are, for example, glycerol, polyglycerol, sorbitol, dimethyl isosorbide, lactic acid and/or lactates, especially sodium lactate, butylene glycol, propylene glycol, biosaccharide gum 1, glycine soya, hydroxyethylurea, ethylhexyloxyglycerol, pyrrolidonecarboxylic acid and urea. In addition, it is especially advantageous to use polymeric moisturizers from the group of water-soluble and/or water-swellable polysaccharides and/or those that can be gelated with the aid of water. Especially advantageous are, for example, hyaluronic acid, chitosan and/or a fucose-rich polysaccharide available under the Fucogel™ 1000 name from SOLABIA S.A.

In the context of the present invention, it is particularly advantageously possible to use water-soluble antioxidants, such as vitamins, e.g. ascorbic acid and derivatives thereof. Vitamin E and derivatives thereof and vitamin A and derivatives thereof are especially advantageous.

Other advantageous active ingredients in the composition according to the invention are α-hydroxy acids such as glycolic acid, lactic acid, malic acid, tartaric acid, citric acid and mandelic acid, β-hydroxy acids such as salicylic acid and acylated derivatives thereof, 2-hydroxyalkanoic acid and derivatives thereof; natural active ingredients and/or derivatives thereof, such as alpha-lipoic acid, folic acid, phytoene, D-biotin, coenzyme Q10, alpha-glucosylrutin, carnitine, carnosine, natural and/or synthetic isoflavonoids, creatine, creatinine, taurine and/or [beta]-alanine and 8-hexadecene-1,16-dicarboxylic acid (dioic acid, CAS number 20701-68-2; INCI name Octadecendioic Acid) and/or licochalcone A and plant extracts.

The cosmetic composition according to the invention may also be used for decorative, in particular color or effect-imparting dressing of human skin, mucosa, semi-mucosa and hair, in particular the eyelids and eyebrows, generally not head hair. The decorative, i.e. color effect or other effect, for example a glitter effect, metallic effect etc., is achieved by at least one effect-imparting, in particular color- and/or effect-imparting constituent. The decorative composition according to the invention can be, for example, a face make-up (foundation), a tinted (day) cream, a blusher, a rouge, a mascara, an eyeliner, a kohl pencil, an eye shadow, a lipstick, a lip gloss, a nail polish, especially a water-based nail polish. A characteristic of the decorative cosmetic compositions is generally that they are so-called “leave on” products, which at least partially remain on the skin or hair after application.

When used as a decorative cosmetic composition, the cosmetic composition according to the invention may in particular be solid, liquid or semi-solid. For example, the composition may be present in the form of oil-in-water, water-in-oil, water-in-silicone-oil, silicone-oil-in-water, oil-in-water-in-oil, water-in-oil-in-water or solid emulsions (emulsions stabilized by solids such as Pickering emulsions). The formulation according to the invention may also be foamed with a propellant gas. The formulation according to the invention can also be in the form of “loose powder”, compact powder, foam (so-called mousse), sticks or in the form of the aforementioned liquid or viscous emulsions.

The composition according to the invention preferably comprises at least one effect-imparting constituent. The constituents mentioned may especially have a colouring effect or else provide other effects, such as sparkle and/or metallic effects. The composition according to the invention preferably comprises at least one dye which is preferably selected from the group of lipophilic dyes, hydrophilic dyes, pigments and nacre. Particularly advantageously in accordance with the invention, the concentration of dyes is 0.01% to 40% by weight, particularly advantageously 1.0% to 30% by weight, very particularly advantageously from 2.0% to 25% by weight, based in each case on the total weight of the composition.

For example, it is possible to use lipophilic dyes, such as Sudan I (yellow), Sudan II (orange), Sudan III (red), Sudan IV (scarlet), DC Red 17, DC Green 6, (3-carotene, soya oil, DC Yellow 11, DC Violet 2, DC Orange 5 and DC Yellow 10.

The pigments may in principle be any inorganic or organic pigments which are used in cosmetic or dermatological compositions. The pigments used in accordance with the invention may, for example, be white or coloured, and they may be encased or coated with a hydrophobic coating composition or be uncoated.

Advantageously, the pigments are selected from the group of the metal oxides, such as the oxides of iron (especially the oxides that are yellow, red, brown or black in colour), titanium dioxide, zinc oxide, cerium oxide, zirconium oxide, chromium oxide; manganese violet, ultramarine blue, Prussian blue, ultramarine and iron blue, bismuth oxide chloride, mother of pearl, mica pigments coated with titanium or bismuth oxide chloride, coloured pearlescent pigments, for example titanium-mica pigments comprising iron oxides, titanium-mica pigments, especially comprising iron blue or chromium oxide, titanium-mica pigments comprising an organic pigment of the aforementioned type, and pearlescent pigments based on bismuth oxide chloride, carbon black, the pigments of the D&C type, and the coating materials based on cochineal red, barium, strontium, calcium and aluminium, and mixtures thereof.

Particularly advantageously used are the pigments of iron oxides or titanium dioxide.

For better wettability of the pigments by the fatty phase oils, the surface of the pigments is preferably treated with a hydrophobic coating composition. The hydrophobic coating composition is preferably selected from the group of silicones, such as methicone, dimethicone, perfluoroalkylsilanes; fatty acids such as stearic acid; metal soaps such as aluminum dimyristate, the aluminum salt of hydrogenated tallow glutamate, perfluoroalkyl phosphates, perfluoroalkylsilanes, perfluoroalkylsilazanes, hexafluoropropylene polyoxides, polyorganosiloxanes comprising perfluoroalkyl perfluoropolyether groups, amino acids; N-acylated amino acids or salts thereof; lecithin, isopropyl triisostearyl titanate and mixtures thereof. The N-acylated amino acids may comprise an acyl group having 8 to 22 carbon atoms, for example 2-ethylhexanoyl, caproyl, lauroyl, myristoyl, palmitoyl, stearoyl or cocoyl. The salts of these compounds may be aluminum salts, magnesium salts, calcium salts, zirconium salts, tin salts, sodium salts or potassium salts. The amino acid may be, for example, lysine, glutamic acid or alanine.

If the cosmetic composition according to the invention is used as a hair-setting composition, it may, in the context of the present invention, be in the form of a spray, a foam, a gel, an emulsion, a solution or a cream, such as a mousse, liquid setting agent, hairspray, styling gel, styling cream, foam aerosol etc.

When using the cosmetic composition according to the invention as a hair-setting composition, it preferably comprises 0.1 to 20% by weight of the polyurethane described above and in particular 0.5 to 10% by weight, based in each case on the total weight of the composition.

In addition to the polyurethane described above, the composition of the invention may comprise further suitable film formers, which in particular may also contribute to the setting and styling of hair.

The concentration of one or more further film formers may be 0 to 20% by weight and in particular 0 to 10% by weight, based in each case on the total weight of the composition.

Advantageously, the film former(s) are selected from the group of water-soluble or water-dispersible polyurethanes other than the polyurethanes used in accordance with the invention, the polyureas, silicone resins and/or polyesters, and the non-ionic, anionic, amphoteric and/or cationic polymers and mixtures thereof. Film formers which are suitable and preferred according to the invention are known per se to those skilled in the art and are described, for example, in WO 2009/118105 A1.

The hair-setting composition comprises, in particular, water and optionally a cosmetically suitable solvent. Preferred solvents are aliphatic alcohols having C2-4 carbon atoms such as ethanol, isopropanol, t-butanol, n-butanol; polyols such as propylene glycol, glycerol, ethylene glycol, and polyol ethers; acetone; unbranched or branched hydrocarbons such as pentane, hexane, isopentane, and cyclic hydrocarbons such as cyclopentane and cyclohexane; and mixtures thereof. An especially preferred solvent is ethanol. However, the content of such solvents is preferably less than 80% by weight, more preferably less than 55% by weight, even more preferably less than 40% by weight, in accordance with the fact that, according to the invention, the hair-setting compositions are preferably low in VOCs. In particular, the water content may be in the range, for example, of 20 to 94% by weight, preferably of 30 to 80% by weight, more preferably of more than 45 to 70% by weight, based on the total weight of the composition. The medium is advantageously an aqueous-alcoholic mixture. The proportion of alcohol in the mixture is in the range of 0 to 90% by weight, preferably 0 to 70% by weight, more preferably 0 to 55% by weight, even more preferably 0 to 40% by weight, based on the total weight the composition.

The cosmetic composition according to the invention can generally be prepared by any method known to those skilled in the art, for example by mixing and/or dispersing the individual components.

The present invention therefore also relates to a process for producing the cosmetic composition according to the invention by mixing and/or dispersing the individual components.

The present invention also relates the use of a cosmetic composition according to any of the claims for application to the skin and/or hair and/or nails, preferably to facial skin.

In particular, the present invention relates to the use according to the invention, wherein a film forms on the skin and/or hair and/or nails, preferably facial skin, after application. In this case, in accordance with the invention, it is preferably a so-called peel-off mask. After application to the skin, the cosmetic composition according to the invention forms as continuous a film as possible, which remains on the skin for a certain time, for example 1 to 60 minutes, in order to develop the cosmetic effect, in particular the skin-care effect, and to dry. After this time has elapsed, the film on the skin can be peeled off the skin by the user as a film, if possible in one piece, wherein the film is peeled off preferably without leaving any residue. A cleansing effect is also created by the film sticking to the skin and impurities and/or horny skin are removed when the film is peeled off. Further details on peel-off masks are known per se to those skilled in the art and, for example, in WO 2009/118104 A1. The cosmetic composition according to the invention can also be used as a nail varnish, in which case the nail varnish can be removed from the nails, preferably as a whole, so that according to the invention preferably no nail polish removers, which may contain organic solvents, have to be used.

The present invention also relates to a cosmetic process for cleansing and caring for the skin and/or of hair and/or of nails and/or for applying a decorative effect to the skin and/or hair and/or nails, comprising applying the cosmetic composition according to the invention to the skin and/or hair and/or nails and optionally thereafter removing the composition from the skin and/or hair and/or nails.

With regard to the process according to the invention and the use according to the invention, what has already been stated with regard to the cosmetic composition according to the invention applies accordingly.

The present invention is elucidated using examples, which are not to be understood as being limiting. All amounts, proportions and percentages, unless stated otherwise, are based on weight and the total amount or total weight of the compositions.

### EXAMPLES

The aqueous polyurethane dispersions PU1 to PU5 listed in Table 1 are prepared as follows: Aqueous polyurethane dispersion PU 1:

318.8 g of a polyester composed of adipic acid, hexanediol and neopentyl glycol having a number-average molecular weight of 1700 g/mol were heated to 65° C. Then 87.9 g of Desmodur® W were added and the mixture was stirred at 125° C. until the NCO content had fallen below the theoretical NCO content. The molar ratio of isocyanate groups to hydroxyl groups in the prepolymer step was 1.79. The finished prepolymer was dissolved in 720 g of acetone at 50° C. and then a solution of 33.9 g of diaminosulfonate, 1.6 g of ethylenediamine and 102 g of water was added. The post-stirring time was 15 min. The mixture was then dispersed by addition of 515 g of water. The solvent was removed by distillation under reduced pressure and a storage-stable dispersion was obtained. The solids content was adjusted to 40% by weight by adding water.

Aqueous polyurethane dispersion PU 2:

1360.0 g of a polyester composed of adipic acid, hexanediol and neopentyl glycol having an average molecular weight of 1700 g/mol were heated to 65° C. Subsequently, 318.5 kg of isophorone diisocyanate (IPDI) were added and the mixture was stirred at 105° C. until the NCO value had gone below the theoretical value. The finished prepolymer was dissolved with 3000 g of acetone at 50° C. and then a solution of 23.4 g of isophoronediamine (IPDA), 129.6 g of diaminosulfonate and 357 g of water was metered in. The post-stirring time was 15 min. The mixture was then dispersed by addition of 2900 g of water. Subsequently, the solvent was removed by distillation under reduced pressure, and a storage-stable dispersion was obtained; the solids content was adjusted by addition of water.

Solids content: 32% by weight

Particle size (LCS): 27 nm

Viscosity: 1500 mPas

pH: 7.3

Aqueous polyurethane dispersion PU 3:

1649.0 g of a polyester composed of adipic acid, hexanediol and neopentyl glycol having an average molecular weight of 1700 g/mol (component A2)) were heated to 65° C. Subsequently, 291.7 kg of hexamethylene diisocyanate (component A1)) were added and the mixture was stirred at 100 to 115° C. until the NCO value had gone below the theoretical value. The finished prepolymer was dissolved with 3450 g of acetone at 50° C. and then a solution of 16.8 g of ethylenediamine (component B1)), 109.7 g of diaminosulfonate (component B2)) and 425 g of water was metered in. The post-stirring time was 15 min. The mixture was then dispersed by addition of 1880 g of water.

This was followed by removal of the solvent by distillation under reduced pressure to obtain a storage-stable dispersion.

Solids content: 42% by weight

Particle size (LCS): 168 nm

Viscosity: 425 mPas

pH: 7.07

Aqueous polyurethane dispersion PU 4:

340 g of a polyester composed of adipic acid, hexanediol and neopentyl glycol having an average molecular weight of 1700 g/mol (component A2)) were heated to 65° C. Subsequently, 60.1 kg of hexamethylene diisocyanate (component A1)) were added and the mixture was stirred at 105° C. until the NCO value had gone below the theoretical value. The finished prepolymer was dissolved with 711 g of acetone at 50° C. and then a solution of 2.1 g of ethylenediamine (component B1)), 32.4 g of diaminosulfonate (component B2)) and 104.3 g of water was metered in. The post-stirring time was 15 min. The mixture was then dispersed by addition of 1880 g of water. This was followed by removal of the solvent by distillation under reduced pressure to obtain a storage-stable dispersion.

Solids content: 40% by weight

Particle size (LCS): 198 nm

Viscosity: 700 mPas

pH: 6.31

Aqueous polyurethane dispersion PU 5:

450 g of PolyTHF® 1000 (component A2)) and 2100 g of PolyTHF® 2000 (component A2)) were heated to 70° C. Subsequently, a mixture of 225.8 g of hexamethylene diisocyanate (component A1)) and 298.4 g of isophorone diisocyanate (component A1)) was added, and the mixture was stirred at 100 to 115° C. until the NCO value had gone below the theoretical value. The finished prepolymer was dissolved with 5460 g of acetone at 50° C. and then a solution of 351 g of diaminosulfonate (component B2)) and 610 g of water was metered in. The post-stirring time was 15 min. The mixture was then dispersed by addition of 1880 g of water. This was followed by removal of the solvent by distillation under reduced pressure to obtain a storage-stable dispersion.

Solids content: 40% by weight

Viscosity: 1370 mPas

The cosmetic compositions 1, 1.1, 1.2, 1.3, 2, 3, 4, 5, 5.1, 6 and 7 according to the invention and the comparative compositions V1, V2, V3, V4, V5, V6, V8, V9, V10, V11, V12, V13, V14 and V15 shown in Table 1 are prepared from the polyurethane dispersions PU 1 to 5 described according to the following general procedures.

3-Phenylpropanol and/or 1,2-octanediol were added to 2-methyl-1,3-propanediol and dissolved with stirring on a magnetic stirrer with a suitable magnetic stirrer bar in order to prepare the solutions. These solutions are then added to the aqueous polyurethane dispersions at the desired concentration and mixed.

TABLE 1

| Experiment | |
|---|---|
| number | Composition of the tested dispersion |
| C1 | PU 1 |
| C2 | PU 2 |
| C3 | PU 3 |
| C4 | PU 4 |
| C5 | PU 5 |
| 1 | PU 1 + 1.5% by weight of a solution consisting of 81.7% by weight |
| | 2-methyl-1,3-propanediol, 15% by weight 1,2-octanediol |
| | (caprylyl glycol) and 3.3% by weight 3-phenylpropanol |
| 1.1 | PU 1 + 1.5% by weight 2-methyl-1,3-propanediol |
| 1.2 | PU 1 + 1.5% by weight of a solution consisting of 30% by weight |
| | 1,2-octanediol (caprylyl glycol) and 70% by weight 2-methyl-1,3-propanediol |
| 1.3 | PU 1 + 1.5% by weight of a solution consisting of 5% by weight |
| | 3-phenylpropanol and 95% by weight 2-methyl-1,3-propanediol |
| 2 | PU 2 + 1.5% by weight of a solution consisting of 81.7% by weight |
| | 2-methyl-1,3-propanediol, 15% by weight 1,2-octanediol |
| | (caprylyl glycol) and 3.3% by weight 3-phenylpropanol |
| 3 | PU 3 + 1.5 AD |
| 4 | PU 4 + 1.5 AD |
| 5 | PU 5 + 1.5% by weight of a solution consisting of 81.7% by weight |
| | 2-methyl-1,3-propanediol, 15% by weight 1,2-octanediol |
| | (caprylyl glycol) and 3.3% by weight 3-phenylpropanol |
| 5.1 | PU 5 + 1.5% by weight 2-methyl-1,3-propanediol |
| 6 | PU 1 + 0.5% by weight of a solution consisting of 81.7% by weight |
| | 2-methyl-1,3-propanediol, 15% by weight 1,2-octanediol |
| | (caprylyl glycol) and 3.3% by weight 3-phenylpropanol |
| 7 | PU 1 + 5.0% by weight of a solution consisting of 81.7% by weight |
| | 2-methyl-1,3-propanediol, 15% by weight 1,2-octanediol |
| | (caprylyl glycol) and 3.3% by weight 3-phenylpropanol |
| C8 | Dermacryl ® C, INCI Name: Acrylate Copolymer (Nouryon) |
| C9 | Dermacryl ® C, INCI Name: Acrylate Copolymer (Nouryon) + 1.5% by weight |
| | of a solution consisting of 81.7% by weight 2-methyl-1,3-propanediol, 15% by |
| | weight 1,2-octanediol (caprylyl glycol) and 3.3% by weight 3-phenyl-propanol |
| C10 | PU 2 + 1.0% by weight ethylene glycol |
| C11 | PU 2 + 0.5% by weight ethylene glycol |
| C12 | PU 2 + 1.0% by weight propylene glycol |
| C13 | PU 2 + 0.5% by weight propylene glycol |
| C14 | PU 1 + 1.5% by weight ethylene glycol |
| C15 | PU 1 + 1.5% by weight propylene glycol |

The amounts stated are each based on the total dispersion.

C Comparative Experiment

3. Method of Measurement

In each case, polymeric films are prepared with the dispersions specified in Table 1. For this purpose, a film is applied to release paper with the aid of a slotted doctor knife. The doctor knife is placed on the paper. In front of the slotted doctor blade, the liquid solution is applied to the paper with a pipette. A thin film, ca. 0.5 mm thick, is applied between the gap and the paper by peeling it off. This film is dried for 24 hours until it is no longer damp and is completely cured. Sample of ca 4 mm length are cut to generate hysteresis curves. A ZwickiZ2.5 tensiometer from Zwick Roell was used for the measurements.

The hysteresis measurements were carried out as follows:

Elongation 100% (see Tables 2, 3 and 4):

A load is applied to the samples to lengthen them to 100% elongation at a rate of 50 mm/min. Then the load is removed until the specimen has assumed the length of the original length—remaining elongated. This procedure is repeated ten times with each sample.

Elongation 500% (see Tables 5, 6 and 7):

A load is applied to the specimens to elongate them stepwise to a lengthening of 50 to 500% at a rate of 50 mm/min. After each lengthening, the load is removed until the specimen has assumed the length of the original length—remaining elongated.

TABLE 2
Remaining elongation 100%, the remaining elongation after up to 10 times elongating
to 100% is shown in %, in each case based on the original sample length

| Number of | No. | | | | | | | | |
|---|---|---|---|---|---|---|---|---|---|
| elongations | C1 | 1 | Δ to C1 | C2 | 2 | Δ to C2 | C5 | 5 | Δ to C5 |
| 1 | 21.12 | 13.10 | 8.02 | 10.15 | 7.15 | 3.40 | 9.90 | 6.45 | 3.45 |
| 2 | 23.59 | 15.12 | 8.47 | 12.57 | 9.17 | 3.39 | 11.90 | 7.79 | 4.11 |
| 3 | 25.01 | 16.28 | 8.72 | 13.84 | 10.28 | 3.56 | 13.01 | 8.59 | 4.41 |
| 4 | 26.01 | 17.23 | 8.78 | 14.57 | 11.06 | 3.50 | 13.87 | 9.29 | 4.58 |
| 5 | 26.76 | 17.82 | 8.94 | 15.35 | 11.88 | 3.47 | 14.65 | 9.73 | 4.92 |
| 6 | 27.45 | 18.31 | 9.14 | 15.84 | 12.34 | 3.50 | 15.17 | 10.07 | 5.11 |
| 7 | 27.82 | 18.81 | 9.00 | 16.40 | 12.76 | 3.64 | 15.65 | 10.43 | 5.22 |
| 8 | 28.23 | 19.21 | 9.03 | 16.79 | 13.27 | 3.52 | 16.06 | 10.65 | 5.42 |
| 9 | 28.70 | 19.51 | 9.19 | 17.12 | 13.54 | 3.58 | 16.42 | 10.90 | 5.53 |
| 10 | 28.98 | 19.87 | 9.10 | 17.65 | 13.90 | 3.74 | 16.76 | 11.09 | 5.67 |

C comparative experiment

TABLE 3
Remaining elongation 100%, the remaining elongation after up to 10 times elongating
to 100% is shown in %, in each case based on the original sample length

| Number of | No. | | | | | | | | | |
|---|---|---|---|---|---|---|---|---|---|---|
| elongations | C1 | 1.1 | Δ to C1 | 1.2 | Δ to C1 | 1.3 | Δ to C1 | C5 | 5.1 | Δ to C5 |
| 1 | 21.12 | 12.84 | 8.28 | 12.01 | 9.11 | 13.2 | 7.92 | 9.9 | 7.79 | 2.11 |
| 2 | 23.59 | 15.01 | 8.58 | 14.28 | 9.31 | 15.32 | 8.27 | 11.9 | 9.40 | 2.50 |
| 3 | 25.01 | 16.2 | 8.81 | 15.67 | 9.34 | 16.65 | 8.36 | 13.01 | 10.37 | 2.64 |
| 4 | 26.01 | 17.06 | 8.95 | 16.62 | 9.39 | 17.53 | 8.48 | 13.87 | 11.12 | 2.75 |
| 5 | 26.76 | 17.76 | 9.00 | 17.23 | 9.53 | 18.43 | 8.33 | 14.65 | 11.73 | 2.92 |
| 6 | 27.45 | 18.37 | 9.08 | 18.12 | 9.33 | 18.92 | 8.53 | 15.17 | 12.17 | 3.00 |
| 7 | 27.82 | 18.76 | 9.06 | 18.59 | 9.23 | 19.45 | 8.37 | 15.65 | 12.59 | 3.06 |
| 8 | 28.23 | 19.29 | 8.94 | 19.03 | 9.2 | 19.95 | 8.28 | 16.06 | 12.84 | 3.22 |
| 9 | 28.7 | 19.67 | 9.03 | 19.46 | 9.24 | 20.42 | 8.28 | 16.42 | 13.15 | 3.27 |
| 10 | 28.98 | 19.99 | 8.99 | 19.85 | 9.13 | 20.73 | 8.25 | 16.76 | 13.42 | 3.34 |

C comparative experiment

TABLE 4
Remaining elongation 100%, the remaining elongation after up to 10 times elongating
to 100% is shown in %, in each case based on the original sample length

| | No. | | | | | | | | | | | | | |
|---|---|---|---|---|---|---|---|---|---|---|---|---|---|---|
| | | | | | | | | Δ | | | | | | |
| | | | | | | | | between | | | | | | |
| Number of | | | | | | | | C8 and | | | | | | |
| elongations | C1 | 6 | Δ to C1 | 7 | Δ to C1 | C8 | C9 | C9 | C10 | C11 | C12 | C13 | C14 | C15 |
| 1 | 21.12 | 18.79 | 2.33 | 9.70 | 11.42 | 36.71 | 39.62 | −2.91 | 14.96 | 17.40 | 12.02 | 12.51 | 13.66 | 13.26 |
| 2 | 23.59 | 21.34 | 2.25 | 11.84 | 11.75 | 41.56 | 44.70 | −3.14 | 19.20 | 22.24 | 15.46 | 15.23 | 16.01 | 15.52 |
| 3 | 25.01 | 22.98 | 2.03 | 13.29 | 11.72 | 43.9 | 47.17 | −3.27 | 21.70 | 24.78 | 16.96 | 17.51 | 17.47 | 16.84 |
| 4 | 26.01 | 23.98 | 2.03 | 14.29 | 11.72 | 46.56 | 49.78 | −3.22 | 23.60 | 27.63 | 18.69 | 18.81 | 18.52 | 17.87 |
| 5 | 26.76 | 24.71 | 2.05 | 15.29 | 11.47 | 47.88 | 51.26 | −3.38 | 25.05 | 29.50 | 19.57 | 19.90 | 19.40 | 18.72 |
| 6 | 27.45 | 25.48 | 1.97 | 15.96 | 11.49 | 48.24 | 52.45 | −4.21 | 26.25 | 30.74 | 20.44 | 20.80 | 20.15 | 19.46 |
| 7 | 27.82 | 25.98 | 1.84 | 16.59 | 11.23 | 50.03 | 53.76 | −3.73 | 27.26 | 32.47 | 21.00 | 21.63 | 20.75 | 20.09 |
| 8 | 28.23 | 26.37 | 1.86 | 17.23 | 11.00 | 50.51 | 53.81 | −3.30 | 28.24 | 33.46 | 21.73 | 22.62 | 21.22 | 20.65 |
| 9 | 28.7 | 26.82 | 1.88 | 17.76 | 10.94 | 51.4 | 54.73 | −3.33 | 28.92 | 34.11 | 22.38 | 23.18 | 21.66 | 20.95 |
| 10 | 28.98 | 27.18 | 1.8 | 18.29 | 10.69 | 51.95 | 55.45 | −3.5 | 29.59 | 34.79 | 22.94 | 23.72 | 22.10 | 21.25 |

C comparative experiment

TABLE 5
Remaining elongation 500%, the remaining elongation is shown after increasing elongation
in 50% steps up to 500%, in each case based on the original sample length

| Elongation | No. | | | | | | | | | | | | | | |
|---|---|---|---|---|---|---|---|---|---|---|---|---|---|---|---|
| [%] | C1 | 1 | Δ to C1 | C2 | 2 | Δ to C2 | C3 | 3 | Δ to C3 | C4 | 4 | Δ to C4 | C5 | 5 | Δ to C5 |
| 50 | 11.01 | 6.23 | 4.78 | 3.23 | 4.15 | −0.92 | 2.76 | 1.04 | 1.73 | 3.42 | −0.17 | 4.00 | 4.54 | 4.01 | 0.13 |
| 100 | 21.17 | 12.73 | 8.44 | 8.54 | 8.12 | 0.42 | 5.70 | 3.48 | 2.22 | 6.70 | 2.92 | 3.78 | 9.26 | 7.54 | 1.72 |
| 150 | 30.45 | 18.90 | 11.55 | 13.93 | 12.15 | 1.78 | 8.87 | 6.45 | 2.42 | 10.65 | 6.89 | 3.75 | 14.23 | 10.98 | 3.25 |
| 200 | 38.98 | 25.12 | 13.86 | 19.29 | 16.29 | 3.00 | 11.92 | 9.48 | 2.45 | 14.68 | 11.37 | 3.31 | 19.23 | 14.54 | 4.69 |
| 250 | 48.06 | 31.45 | 16.61 | 24.59 | 20.14 | 4.06 | 15.23 | 12.73 | 2.50 | 18.87 | 15.59 | 3.28 | 24.45 | 17.95 | 6.50 |
| 300 | 57.34 | 38.26 | 19.09 | 30.29 | 25.12 | 5.16 | 18.90 | 15.73 | 3.17 | 23.13 | 19.53 | 3.59 | 29.65 | 20.92 | 8.72 |
| 350 | 66.87 | 46.17 | 20.69 | 35.93 | 29.92 | 6.01 | 23.09 | 19.17 | 3.92 | 27.93 | 24.04 | 3.89 | 35.09 | 23.62 | 11.47 |
| 400 | 76.70 | 53.53 | 23.17 | 41.93 | 35.04 | 6.89 | 27.88 | 22.90 | 4.98 | 32.76 | 28.56 | 4.20 | 40.81 | 26.49 | 14.33 |
| 450 | 86.76 | 61.81 | 24.95 | 48.37 | 40.19 | 7.78 | 33.65 | 27.24 | 6.41 | 37.95 | 33.81 | 4.14 | 46.49 | 29.48 | 17.01 |
| 500 | 96.54 | 70.11 | 26.02 | 55.18 | 46.26 | 8.93 | 39.59 | 31.76 | 7.83 | 43.82 | 39.18 | 4.63 | 53.15 | 32.87 | 20.28 |

C comparative experiment

TABLE 6
Remaining elongation 500%, the remaining elongation is shown after increasing elongation
in 50% steps up to 500%, in each case based on the original sample length

| | No.: | | | | | | | | | |
|---|---|---|---|---|---|---|---|---|---|---|
| Elongation | | | | | | | | C5 | | |
| [%] | C1 | 1.1 | Δ to C1 | 1.2 | Δ to C1 | 1.3 | Δ to C1 | PU 5 | 5.1 | Δ to C5 |
| 50 | 11.01 | 6.59 | 4.42 | 5.87 | 5.14 | 6.45 | 4.56 | 4.54 | 4.59 | −0.05 |
| 100 | 21.17 | 13.65 | 7.52 | 12.02 | 9.15 | 13.37 | 7.80 | 9.26 | 8.62 | 0.64 |
| 150 | 30.45 | 20.53 | 9.92 | 18.12 | 12.33 | 20.12 | 10.33 | 14.23 | 13.01 | 1.22 |
| 200 | 38.98 | 27.56 | 11.42 | 24.4 | 14.58 | 27.09 | 11.89 | 19.23 | 17.26 | 1.97 |
| 250 | 48.06 | 34.76 | 13.3 | 30.98 | 17.08 | 34.42 | 13.64 | 24.45 | 21.59 | 2.86 |
| 300 | 57.34 | 42.33 | 15.01 | 37.98 | 19.36 | 41.96 | 15.38 | 29.65 | 25.62 | 4.03 |
| 350 | 66.87 | 50.06 | 16.81 | 45.51 | 21.36 | 49.67 | 17.20 | 35.09 | 29.78 | 5.31 |
| 400 | 76.7 | 59.12 | 17.58 | 53.51 | 23.19 | 57.96 | 18.74 | 40.81 | 33.67 | 7.14 |
| 450 | 86.76 | 68.24 | 18.52 | 62.04 | 24.72 | 67.4 | 19.36 | 46.49 | 37.93 | 8.56 |
| 500 | 96.54 | 77.65 | 18.89 | 71.87 | 24.67 | 76.87 | 19.67 | 53.15 | 42.51 | 10.64 |

C comparative experiment

TABLE 7
Remaining elongation 500%, the remaining elongation is shown after increasing elongation
in 50% steps up to 500%, in each case based on the original sample length

| Elongation | No.: | | | | | | | | | | |
|---|---|---|---|---|---|---|---|---|---|---|---|
| [%] | C1 | 6 | Δ to C1 | 7 | Δ to C1 | C10 | C11 | C12 | C13 | C14 | C15 |
| 50 | 11.01 | 9.51 | 1.50 | 4.84 | 6.17 | 7.65 | 6.98 | 5.58 | 5.42 | 8.78 | 6.74 |
| 100 | 21.17 | 18.37 | 2.80 | 9.51 | 11.66 | 15.66 | 14.9 | 11.28 | 11.27 | 16.52 | 13.38 |
| 150 | 30.45 | 26.95 | 3.50 | 14.76 | 15.69 | 23.81 | 22.26 | 17.28 | 16.81 | 24.07 | 19.94 |
| 200 | 38.98 | 34.9 | 4.08 | 20.37 | 18.61 | 33.05 | 31.17 | 22.88 | 22.68 | 31.58 | 26.45 |
| 250 | 48.06 | 43.4 | 4.66 | 26.59 | 21.47 | 42.37 | 39.64 | 28.00 | 28.46 | 39.86 | 33.45 |
| 300 | 57.34 | 51.92 | 5.42 | 33.17 | 24.17 | 51.87 | 48.42 | 33.46 | 34.43 | 47.84 | 40.57 |
| 350 | 66.87 | 60.87 | 6.00 | 40.14 | 26.73 | 61.77 | 56.36 | 38.27 | 39.19 | 56.34 | 48.39 |
| 400 | 76.7 | 70.12 | 6.58 | 47.26 | 29.44 | 72.89 | 65.02 | 44.96 | 45.07 | 65.54 | 56.65 |
| 450 | 86.76 | 79.87 | 6.89 | 55.12 | 31.64 | 81.57 | 72.4 | 49.93 | 51.27 | 75.33 | 65.21 |
| 500 | 96.54 | 90.12 | 6.42 | 63.23 | 33.31 | — | — | — | — | | |

C comparative experiment

1. A cosmetic composition, comprising at least one aqueous polyurethane dispersion and 0.1 to 7.5% by weight, based on the at least one aqueous polyurethane dispersion, of a mixture comprising at least one alkanediol.

2. The cosmetic composition as claimed in claim 1,
wherein the at least one alkanediol is selected from the group consisting of 2-methyl-1,3-propanediol (CAS 2163-42-0), 1,2-octanediol (caprylyl glycol, CAS 1117-86-8), isomers thereof and mixtures thereof.

3. The cosmetic composition as claimed in claim 1,
wherein the mixture comprises 2 methyl-1,3-propanediol, 1,2-octanediol (caprylyl glycol), and 3-phenylpropanol.

4. The cosmetic composition as claimed in any of claim 1, wherein the mixture comprises 70 to 89% by weight 2-methyl-1,3-propanediol, 10 to 30% by weight 1,2-octanediol (caprylyl glycol), and 1 to 5% by weight 3-phenylpropanol based on a total weight of the mixture.

5. The cosmetic composition as claimed in any of claim 1, wherein the at least one aqueous polyurethane dispersion comprises at least one polyurethane obtained by reacting one or more water-insoluble, non-water-dispersible, isocyanate-functional polyurethane prepolymers A) with at least one amino-functional compound B).

6. The cosmetic composition as claimed in claim 5,
wherein the prepolymers A) used to produce the at least one polyurethane is obtained by reacting at least one polyisocyanate and one or more polyols selected from the group consisting of polyether polyols, polycarbonate polyols, polyether-polycarbonate polyols, polyester polyols and mixtures thereof.

7. The cosmetic composition as claimed in claim 6,
wherein the at least one polyisocyanate is selected from the group consisting of 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof of any desired isomer content, 1,4-cyclohexylene diisocyanate, 4-isocyanatomethyl-1,8-octane diisocyanate (nonane triisocyanate), 1,4-phenylene diisocyanate, 2,4- and/or 2,6-tolylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI) and alkyl-2,6-diisocyanatohexanoates (lysine diisocyanates) having C₁-C₈-alkyl groups, and mixtures thereof.

8. The cosmetic composition as claimed in any of claim 1, wherein the at least one aqueous polyurethane dispersion is present to an extent of 1 to 90% by weight, based on the solids content of the composition.

9. The cosmetic composition as claimed in any of claim 5, wherein the at least one amino-functional compound B) comprises at least one diamine.

10. The cosmetic composition as claimed in any of claim 5, wherein the at least one amino-functional compound B) comprises at least one amino-functional compound B1) having no ionic and/or ionogenic groups, wherein the at least one amino-functional compound B1) is incorporated into the at least one polyurethane.

11. The cosmetic composition as claimed in any of claim 5, wherein the at least one amino-functional compound B) comprises at least one amino-functional compound B2) having ionic and/or ionogenic groups, wherein the at least one amino-functional compound B2) is incorporated into the at least one polyurethane.

12. A process for the preparing the cosmetic composition as claimed in claim 1 comprising mixing and/or dispersing the at least one aqueous polyurethane dispersion and the mixture.

13. A method of forming a film on the skin and/or hair and/or nails after application, comprising applying the cosmetic composition as claimed in claim 1 to the skin and/or hair and/or nails.

14. A cosmetic process for cleansing and caring for the skin and/or of hair and/or of nails and/or for applying a decorative effect to the skin and/or hair and/or nails, comprising applying the cosmetic composition as claimed in claim 1 to the skin and/or hair and/or nails and optionally thereafter removing the composition from the skin and/or hair and/or nails.
